(19) United States
(12) Patent Application Publication
(10) Pub. No.: US 2005/0256093 A1
(43) Pub. Date: Nov. 17, 2005

(54) Branched polyamine steroid derivatives

(75) Inventor: Tore Duvold, Frederiksberg C (DK)

Correspondence Address: BIRCH STEWART KOLASCH & BIRCH, PO BOX 747, FALLS CHURCH, VA, 22040-0747, US

(73) Assignee: Leo Pharma, Ballerup (DK)

(21) Appl. No.: 10/509,911

(22) PCT Filed: Apr. 4, 2003
(86) PCT No.: PCT/DK03/00220

§ 371 Date: Jun. 1, 2005

Related U.S. Application Data
(60) Provisional application No. 60/369,864, filed on Apr. 5, 2002.

(51) Int. Cl. A61K 31/56; C07J 9/00

(52) U.S. Cl. 514/169; 552/554

(57) ABSTRACT

Novel compounds comprising a steroid backbone coupled to a branched polyamine according to formula I 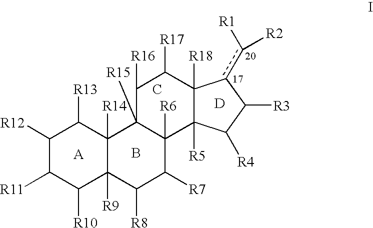 are provided. The compounds show antimicrobial activity and may be used in the treatment and prevention of infections, in particular bacterial infections.

### FIELD OF INVENTION

The present invention relates to novel compounds with a broad spectrum of antimicrobial activity, namely steroids comprising branched polyamine side chains, and to the use of such compounds as antimimcrobial agents in the treatment of infections.

### BACKGROUND OF THE INVENTION

In the field of antibiotics, drug resistance is an ever-increasing problem posing a serious threat to public health. The general belief for many years that infectious diseases could be controlled by the current arsenal of antibacterial drugs has resulted in the development of fewer new and more efficient drugs. Recent widespread emergence of multiple resistance among pathogenic bacteria, however, has sparked renewed interest in the discovery of new antibiotics. Although resistance to many antibiotics such as beta-lactams, macrolides, tetracyclines and aminoglycosides, and the rapid spread of resistance has been recognised for many years, it was assumed that reserve drugs like glycopeptides and fluoroquinolones were sufficient to'combat most infections. However, the many alarming reports of vancomycin-resistance, multiple drug resistance and examples of transfer of resistance genes between different species in the late 1980s and early 1990s has brought the issue of drug resistance to the attention of health authorities and the pharmaceutical industry. It remains an important task to identify new compounds with antimicrobial activity.

Steroids is a group of compounds ubiquitous in living organisms, the prime example of which is hormones. All steroids share a common backbone or nucleus comprising three hexagonal rings and one pentagonal ring, and may thus be referred to as a cyclopentanoneperhydrophenanthrene. Steroids are of pivotal biological importance. They critically influence the catabolism and anabolism of all major biochemical compounds, such as proteins, carbohydrates and lipids, and they do so by inducing the synthesis of enzymes controlling the level of said biochemical compounds. Hormones may be classified as estrogens, androgens, progestins, mineralocorticoids and glucocorticoids. They regulate important aspects of all biological activity, e.g. bone and muscle build-up and maintenance, the blood pressure, glucose level in the blood and the development of the sexual characteristics. With this multitude of biological effects steroids, either in the form of hormones or in the form of chemically closely related derivatives, also offer themselves as potential drugs for various diseases. Steroids in general are used in replacement therapy in patients with insufficient generation of steroids; glucocorticoids, both systemically and topically administered, in high levels are used as antiinflammatory and immunosupprepresive agents; estrogenic and progestational steroids are used to treat dysfunctions in the reproductive system and, more frequently, as contraceptives.

A limited number of steroids exhibit antibiotic effect, an example of which is fusidic acid. Fusidic acid, a fermentation product from Fusidium coccineum, has been known since the early 1960s (U.S. Pat. No. 3,072,531). Fusidic acid (e.g. Fucidin®, LEO Pharmaceutical Products Ltd, Denmark) is used clinically in the treatment of infectious diseases, e.g. staphylococal infections, and it is administered both topically and systemically (Kuchers et al., 1997, and references cited therein; Duvold et al 2001, and references cited therein; Christiansen, 1999, and references cited therein). It is generally administered in combination with common antibiotics, such as penicillins, erythromycins or clindamycin.

More recently, a steroidal antibiotic was isolated from the stomach of the dogfish shark, Squalus acanthias (Moore et al., 1993; Rao et al., 2000). The compound, which is based on a steroid backbone comprising a linear polyamine and sulphate functionality, was termed squalamine and was found to have broad-spectrum antibiotic properties against gram-positive and gram-negative bacteria, fungi and protozoa. The use of native squalamine as an antimicrobial agent is disclosed in U.S. Pat. No. 5,192,756. Squalamine has also been prepared by chemical synthesis although the procedure has been found to be rather cumbersome. A number of squalamine mimics and their use as antibiotics are disclosed in WO 00/09137.

Further squalamine mimics comprising polyamine side chains are disclosed in WO 02/14342 as well as in B. Ding et al., J. Med. Chem. 45, 2002, pp. 663-669.

Branched polyamines have not been reported to exert an antibiotic effect in themselves.

### SUMMARY OF THE INVENTION

The present inventor has surprisingly found that steroid derivatives comprising a steroid backbone coupled to a branched polyamine constitute compounds with a wide antimicrobial, and in particular antibacterial activity. The branched polyamine moiety confers antimicrobial activity to non-antimicrobial steroids, and it improves the antimicrobial activity of steroids which themselves exert an antimicrobial activity.

Accordingly, the present invention relates to a compound of formula I 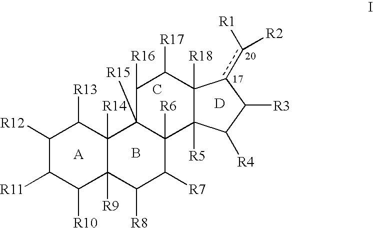
wherein the fused rings A, B, C and D are independently saturated or fully or partially unsaturated;
the bond between C-17 and C-20 is shown with a full and a dotted line to indicate that said bond can be a single or a double bond;
wherein R1 is hydrogen, halogen, a lipophilic group, -(Z)ₙ-(NR-Z)ₚ-N(R)₂ or C(O)-(Z)ₙ-(NR-Z)ₚ-N(R)₂, wherein n is 0 or 1 and p is an integer from 1 and 5;
each Z independently represents straight or branched hydrocarbon diradical, optionally substituted with C₁₋₆ alkyl, C₁₋₆alkenyl, C₁₋₆alkynyl, hydroxy, alkoxy, amino, C₁₋₆aminoalkoxy, C₁₋₆aminoalkyl, C₁₋₆aminoalkylaminocarbonyl, C₁₋₆alkylC₃₋₈cycloalkyl or C₁₋₆alkylheteroaryl;
each R independently represents hydrogen or C₁₋₆alkyl, C₁₋₆aminoalkyl, C₁₋₆aminoalkoxy or C₁₋₆aminoalkylaminocarbonyl, all of which are optionally substituted with alkyl or C₁₋₆aminoalkyl;
provided that at least one Z is substituted with C₁₋₆ alkyl, C₁₋₆alkenyl, C₁₋₆alkynyl, hydroxy, alkoxy, C₁₋₆aminoalkoxy, C₁₋₆aminoalkyl, C₁₋₆aminoalkylaminocarbonyl, C₁₋₆alkylC₃₋₈cycloalkyl or C₁₋₆alkylheteroaryl, or at least one R is different from hydrogen; R2 represents halogen, C₁₋₄alkyl, optionally substituted with COOH; C₁₋₄alkoxy, —COOH, -(Z)ₙ(NR-Z)ₚ-N(R)₂ or C(O)-(Z)ₙ-(NR-Z)ₚ-N(R)₂;
R3 represents hydrogen, halogen or O—R19, wherein R19 represents hydrogen, —SO₃, C₁₋₆alkyl, C₁₋₆acyl or -(Z)ₙ-(NR-Z)ₚ-N(R)₂; each of R4, R7, R8, R10, R11, R12, R13, R16 and R17 independently represent hydrogen, halogen, hydroxy, OSO₃, —O-acyl, -(Z)ₙ-(NR-Z)ₚ-N(R)₂ or C(O)-(Z)ₙ-(NR-Z)ₚ-N(R)₂;
each of R5, R6, R9, R14, R15 and R18 independently represent hydrogen or methyl or are each independently absent when one of the fused rings, A, B, C and D are unsaturated so as to complete the valency of the carbon atom at that site;
provided that at least one, and not more than three of R1, R2, R4, R7, R8, R10, R11, R12, R13, R16 and R17 is -(Z)ₙ-(NR-Z)ₚ-N(R)₂ or C(O)-(Z)ₙ-(NR-Z)ₚ-N(R)₂;
   and pharmaceutically acceptable salts or esters thereof.

The exact mechanism of action of the present compounds is currently unknown. Without wishing to be limited to a particular hypothesis, it is believed that they may perforate cell membranes, and that membrane lysis could occur through pore formation. In this way, the present compounds may be able to circumvent two major drug resistance mechanisms to which some other antibiotics are subject, i.e. enzymatic degradation in the cell and export pathways (Sadownik et al., 1995; Savage and Li, 2000 and references cited therein).

In another aspect, the invention relates to a pharmaceutical composition comprising a compound of formula I together with a pharmaceutically acceptable excipient or diluent.

In a further aspect, the invention relates to the use of a compound of formula I in the manufacture of a medicament for the prevention or treatment of infection.

In a still further aspect, the invention relates to a method of preventing or treating infection, the method comprising administering to a patient in need thereof an effective amount of a compound of formula I.

### BRIEF DESCRIPTION OF THE DRAWINGS

FIG. 1 shows the Minimum Bactericidal Concentration (MBC) for compound 102 with respect to S. aureus.

FIG. 2 shows the Minimum Bactericidal Concentration (MBC) for compound 102 with respect to S. pyogenes.

### DETAILED DESCRIPTION OF THE INVENTION

Definitions

In the present context, the term “hydrocarbon” refers to a compound which solely contains carbon and hydrogen, and in which the carbon atoms form a straight or branched skeleton.

The term “alkyl” is intended to indicate a univalent radical derived from straight or branched alkane by removing a hydrogen atom from any carbon atom. The term includes the subclasses primary, secondary and tertiary alkyl, such as methyl, ethyl, n-propyl, isopropyl, n-butyl, isobutyl, sec.-butyl, tert.-butyl, n-pentyl, isopentyl, n-hexyl and isohexyl.

The term “alkenyl” refers to a univalent radical derived from straight or branched alkene by removing a hydrogen atom from any carbon atom. The term includes the subclasses primary, secondary and tertiary alkenyl, such as vinyl, 1-propenyl, isopropenyl, butenyl, tert.-butenyl, pentenyl and hexenyl.

The term “alkynyl” refers to univalent radical derived from straight or branched alkyne by removing a hydrogen atom from any carbon atom. The term includes ethynyl, propynyl, isopropynyl, tert.-butynyl, pentynyl and hexynyl.

The term “alkoxy” is intended to indicate a radical of formula OR′, wherein R′ is a hydrocarbon radical as defined above, e.g. methoxy, ethoxy, propoxy, butoxy, etc.

The term “alkoxycarbonyl” is intended to indicate a radical of formula —COOR′ wherein R′ is a hydrocarbon radical as defined above, e.g. methoxycarbonyl, ethoxycabonyl, n-propoxycarbonyl, isopropoxycarbonyl, etc.

The term “cycloalkyl” is intended to indicate a saturated cycloalkane radical, e.g. cyclopropyl, cyclobutyl, cyclopentyl or cyclohexyl. Likewise, the term “cycloalkenyl” is intended to indicate cycloalkene radical, e.g. cyclopropenyl, cyclobutenyl, cyclopentenyl or cyclohexenyl.

The term “aryl” is intended to include radicals of carbocyclic aromatic rings, optionally fused bicyclic rings, e.g. phenyl or naphthyl. The term “heteroaryl” is intended to include radicals of heterocyclic aromatic rings, in particular 5- or 6-membered rings with 1-3 heteroatoms selected from O, S and N, or optionally fused bicyclic rings with 1-4 heteroatoms, e.g. pyridyl, tetrazolyl, thiazolyl, imidazolyl, pyrazolyl, oxazolyl, isoxazolyl, thienyl, pyrazinyl, isothiazolyl, benzimidazolyl and benzofuranyl.

The term “acyl” refers to a radical of formula —CO—R′, wherein R′ is a hydrocarbon radical as indicated above.

The term “aralkyl” is intended to indicate an aromatic ring with an alkyl side chain, e.g. benzyl.

The term “halogen” is intended to indicate fluoro, chloro, bromo or iodo.

The term “amino” is intended to indicate a radical of the formula —NR″₂, wherein each R″ independently represnets hydrogen or a hydrocarbon radical.

The term “aminoalkoxy” refers to a radical of formula —OR′—NR″₂, wherein R′ is a hydrocarbon diradical, and each R″ independently represents hydrogen or hydrocarbon radical.

The term “aminoalkyl” refers to a radical of formula —R′—NR″₂, wherein R′ is a hydrocarbon diradical, and each R″ independently represents hydrogen or hydrocarbon radical.

The term “aminoalkylaminocarbonyl” refers to a radical of formula —C(O)—NR″—R′—NR₁₂, wherein R′ is a hydrocarbon diradical, and each R″ independently represents hydrogen or hydrocarbon radical.

The term “branched polyamine” is intended to indicate a compound of the formula NHR-(Z)ₙ-(NR-Z)ₚ-N(R)₂, wherein n and p and each R and Z independently is as previously defined, and wherein at least R is different from hydrogen, and wherein at least one Z is substituted with C₁₋₆alkyl, C₁₋₆alkenyl, C₁₋₆alkynyl, hydroxy, alkoxy, C₁₋₆aminoalkyl, C₁₋₆aminoalkoxy, C₁₋₆aminoalkylaminocarbonyl, C₁₋₆alkylC₃₋₈cycloalkyl or C₁₋₆alkylheteroaryl.

The term “pharmaceutically acceptable salt” is intended to indicate alkali metal or alkaline earth metal salts, for instance sodium, potassium, magnesium or calcium salts, as well as silver salts and salts with bases such as ammonia or suitable non-toxic amines, e.g. lower alkylamines, for instance triethylamine, hydroxy-lower alkylamines, for instance 2-hydroxyethylamine or bis-(2-hydroxyethyl)amine, cycloalkylamines, for instance dicyclohexylamine, or benzylamines, such as N,N′-dibenzylethylenediamine and dibenzylamine, as well as salts with suitable organic or inorganic acids, such as hydrochloric, hydrobromic, hydroiodic, sulfuric, nitric, phosphoric, acetic, lactic, maleic, phtalic, citric, propionic, benzoic, glutaric, gluconic, metanesulfonic, salicylic, succinic, tartaric, toluenesulfonic, sulfamic or fumaric acid.

The term “pharmaceutically acceptable esters” is intended to indicate easily hydrolysable esters such as alkanoyloxyalkyl, aralkanoyloxyalkyl, aroyloxyalkyl, e.g. acetoxymethyl, pivaloyloxymethyl, benzoyloxymethyl esters and the corresponding 1′-oxyethyl derivatives, or alkoxycarbonyloxyalkyl esters, e.g. methoxycarbonyloxymethyl esters and ethoxycarbonyloxymethyl esters and the corresponding 1′-oxyethyl derivatives, or lactonyl esters, e.g. phthalidyl esters, or dialkylaminoalkyl esters, e.g. dimethylaminoethyl esters. Easily hydrolysable esters include in vivo hydrolysable esters of the compounds of formula I. Such esters may be prepared by conventional methods known to persons skilled in the art, such as method disclosed in GB patent No. 1 490 852 incorporated herein by reference.

The terms “antibiotic” and “antimicrobial” are used interchangeably, and are intended to have the same meaning.

### PREFERRED EMBODIMENTS OF THE INVENTION

In an preferred embodiment, R2, R7, R11 and/or R16 represent -(Z)ₙ-(NR-Z)ₚ-N(R)₂ or C(O)-(Z)ₙ-(NR-Z)ₚ-N(R)₂.

Specific examples of R19 are C₁₋₆alkyl and C₁₋₆acyl.

Specific examples of R7, R11 and R16 is —OH. Compounds according to formula I, wherein R11 is O—SO₃ or O-acyl are also believed to be particularly favourable.

A preferred embodiment of the invention relates to a compound of the general formula Ia or 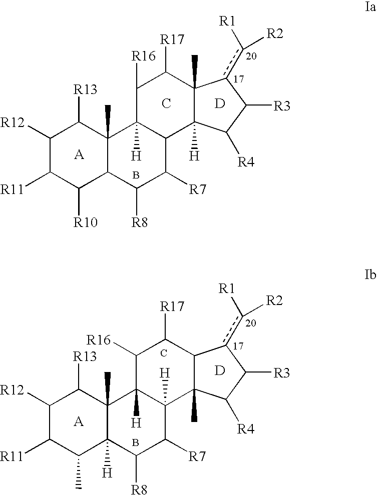
wherein R1, R2, R3, R4, R7, R8, R10, R11, R12, R13, R16 and R17 are as defined previously.

Specific examples of compounds of the invention are compounds of formula Ia or Ib, wherein R2 is -(Z)ₙ-(NR-Z)ₚ-N(R)₂ or C(O)-(Z)ₙ-(NR-Z)ₚ-N(R)₂, especially wherein R7 and R11 are both hydroxy; wherein R11 and R16 are both hydroxy; or wherein R3 is —OR19, wherein R19 is C₁₋₆alkyl or C₁₋₆acyl.

Still more specific examples of compounds of the invention are compounds of formula Ia or Ib, wherein R11 is -(Z)ₙ-(NR-Z)ₚ-N(R)₂ or C(O)-(Z)ₙ-(NR-Z)ₚ-N(R)₂, especially wherein R2 is C₁₋₄alkyl, optionally substituted with COOH; C₁₋₄alkoxy or —COOH; or wherein R3 is —OR19, wherein R19 is C₁₋₆alkyl or C₁₋₆acyl.

In the compounds of formula I, and more specifically of Ia or Ib, R1 is preferably a lipophilic group, i.e. a group which is predominantly non-polar. Non-polar groups at the R1-site are believed to be important for the ability of the compound of the present invention to lodge in a cell membrane which is also lipophilic in nature. Examples of such lipophilic groups are C₁₋₁₀alkyl, aryl, C₃₋₈cycloalkyl, aralkyl with 1-10 carbon atoms in the alkyl moiety, C₁₋₁₀alkylaryl, C₁₋₁₀alkyl-C₃₋₈cycloalkyl, C-₁₋₁₀alkoxy and heteroaryl. Preferably, R1 is a straight or branched, saturated or unsaturated C₁₋₁₀hydrocarbon, e.g. a moiety of formula II 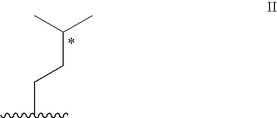
wherein the carbon-carbon bond denoted “*” is a single or double bond.

In a preferred embodiment of the invention, R2 and/or R11 represent a moiety of the formulas VIII, IX, X, XI, XII or XIII as shown below

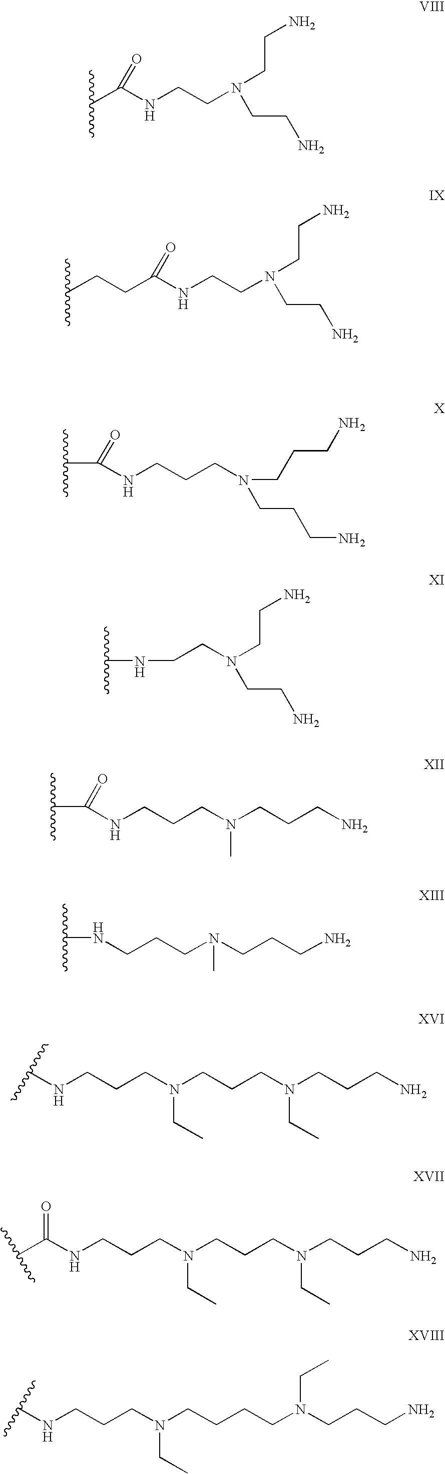

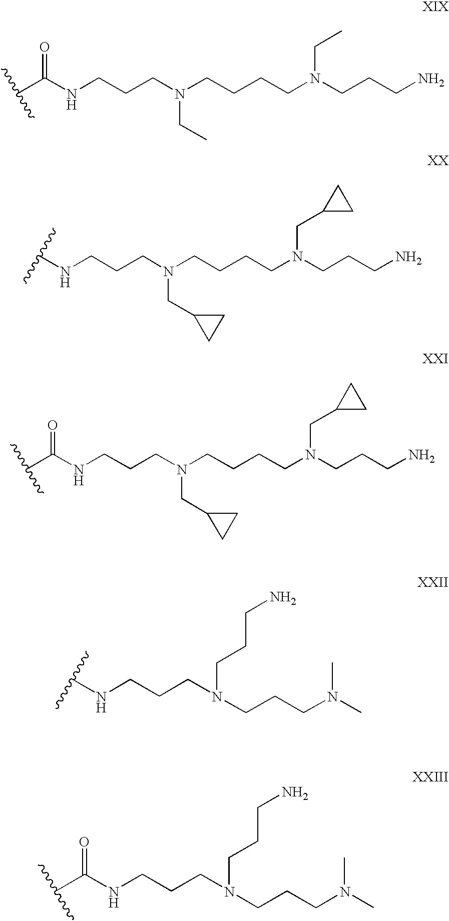

In a particular preferred embodiment, compounds according to formula I are selected from the group consisting of
21-N-{2′-[bis(2′-aminoethyl)amino]ethyl}-17R,20S,24,25-tetrahydrofusid-21-amide (Compound 101)
21-N-{2′-[bis(2′-aminoethyl)amino]ethyl}-11-desoxy-17R,20S,24,25-tetrahydrofusid-21-amide (Compound 102),
21-N-{2′-[bis(2′-aminoethyl)amino]ethyl}-16-desacetoxy-17R,20S,24,25-tetrahydrofusid-21-amide (Compound 103),
21-N-{2′-[bis(2′-aminoethyl)amino]ethyl}-13(17)-en-17,20,24,25-tetrahydrofusidan-21-carboxamide (Compound 104),
21-N-{2′-[bis(2′-aminoethyl)amino]ethyl}-3β-desacetoxy-17R,20S,24,25-tetrahydrofusid-21-amide (Compound 105),
21-N-{2′-[bis(2′-aminoethyl)amino]ethyl}-9(11)-en-17R,20S,24,25-tetrahydrofusid-21-amide (Compound 106),
24-N-{2′-[bis(2′-aminoethyl)amino]ethyl}-3α-hydroxy-5β-cholan-24-amide (Compound 107),
22-N-{2′-[bis(2′-aminoethyl)amino]ethyl}-23,24-bisnor-5-cholenic-22-amide (Compound 108),
21-N-{2′-[bis(2′-aminoethyl)amino]ethyl}-fusid-21-amide (Compound 109),
21-N-{3′-[bis(3′-aminopropyl)amino]propyl}-fusid-21-amide (Compound 110),
21-N-{2′-[bis(2′-aminoethyl)amino]ethyl}-3-OSO₃-11-desoxy-17,20,24,25-tetrahydro-fusid-21-amide (Compound 111),
21-N-{2′-[bis(2′-aminoethyl)amino]ethyl}-11-desoxy-16-desacetoxy-17S,20,24,25-tetrahydrofusid-21-amide (Compound 112),
21-N-{3′-[bis(3′-aminopropyl)amino]propyl}-17R,20S,24,25-tetrahydrofusid-21-amide (Compound 113),
22-N-{3′-[bis(3′-aminopropyl)amino]propyl}-23,24-bisnor-5-cholenic-22-amide (Compound 114),
21-N-{3′-[bis(3′-aminopropyl)amino]propyl}-}-3-OAc-17R,20S,24,25-tetrahydrofusid-21-amide (Compound 115),
21-N-{3′-[bis(3′-aminopropyl)amino]propyl}-}-3-OSO₃-11-desoxy-17,20,24,25-tetrahydrofusid-21-amide (Compound 116),
21-N-{3′-[bis(3′-aminopropyl)amino]propyl}-1-11-desoxy-16-desacetoxy-17S,20,24,25-tetrahydrofusid-21-amide (Compound 117),
3-N-{2′-[bis(2′-aminoethyl)amino]ethyl}-fusidic acid (Compound 118),
21-N-{3′-[(3′-aminopropyl)(methyl)amino]propyl}-17R,20S,24,25-tetrahydrofusid-21-amide (Compound 119),
21-N-{3′-[(3′-aminopropyl)(methyl)amino]propyl}-11-desoxy-17R,20S,24,25-tetrahydrofusid-21-amide (Compound 120),
21-N-{3′-[(3′-aminopropyl)(methyl)amino]propyl}-16-desacetoxy-17R,20S,24,25-tetrahydrofusid-21-amide (Compound 121),
24-N-{3′-[(3′-aminopropyl)(methyl)amino]propyl}-3α-hydroxy-5β-cholan-24-amide (Compound 122),
21-N-{3′-[(3′-aminopropyl)(methyl)amino]propyl}-11-desoxy-16-desacetoxy-17R,20S,24,25-tetrahydrofusid-21-amide (Compound 123),
3-N-{3′-[bis(3′-aminopropyl)amino]propyl}-}-fusidic acid (Compound 124),
3-N-{3′-[(3′-aminopropyl)(methyl)amino]propyl}-fusidic acid (compound 125),
21-N-{3-({4′-[(3′-amino-propyl)-methyl-amino]-butyl}-methyl-amino)-propyl}-17R,20S,24,25-tetrahydrofusid-21-amide (Compound 126),
21-N-{3′-({3′-[(3′-Amino-propyl)-ethyl-amino]-propyl}-ethyl-amino)-propyl}-17R,20S,24,25-tetrahydrofusid-21-amide (Compound 127),
21-N-{3′-({4′-[(3′-amino-propyl)-ethyl-amino]-butyl}-ethyl-amino)-propyl}-17R,20S,24,25-tetrahydrofusid-21-amide (Compound 128),
21-N-3-({3′-[(3′-amino-propyl)-ethyl-amino]-propyl}-ethyl-amino)-propyl}-11-desoxy-17R,20S,24,25-tetrahydrofusid-21-amide (Compound 129),
21-N-{3′-({4′-[(3′-amino-propyl)-cyclopropylmethyl-amino]-butyl}-cyclopropylmethyl-amino)-propyl}-17R,20S,24,25-tetrahydrofusid-21-amide (Compound 130),
21-N-{3′-[(3′-amino-propyl)-(3′-dimethylaminopropyl)-amino]-propyl}-11-desoxy-17R,20S,24,25-tetrahydrofusid-21-amide (Compound 131),
   and pharmaceutically acceptable salts and esters thereof.

Naming of the above mentioned compounds is based on IUPAC for the branched polyamine side chain and on fusidane and steroid conventions for the steroid moiety. Naming has been assisted by using the program available at http://www2.acdlabs.com/ilab/.

Formula I comprise chiral centres as well as carbon-carbon double bonds which allow for stereo and geometric isomers. It is to be understood that the present invention relates to all isomeric and tautomeric forms covered by the formula I, in pure form and as mixtures thereof.

Pharmaceutical Compositions

Compositions of the invention comprise as an active component at least one compound of formula I (hereinafter referred to as the active ingredient) including pharmaceutically acceptable salts and esters thereof together with at least one pharmaceutically acceptable vehicle and/or diluent.

In said composition, the proportion of active ingredient to vehicle may vary from 0.5% to 100% by weight, in particular from about 0.1 to about 50% by weight. The compositions may be prepared in the form of different pharmaceutical formulations such as granulates, tablets, pills, dragees, suppositories, capsules, sustained-release tablets, suspensions, injection and may be filled in bottles or tubes or similar containers in accordance with accepted principles of pharmaceutical formulation, e.g. as disclosed in Remington: The Science and Practice of Pharmacy, 20^{th} Ed., Mack Publishing Company, 2000. Pharmaceutically acceptable organic or inorganic, solid or liquid carriers and/or diluents suitable for oral, enteral, parenteral or topical administration can be used to make up compositions containing the present compounds: water, gelatin, lactose, starch, magnesium stearate, talc, vegetable and animal oils and fats, benzyl alcohol, gum, polyalkylene glycol, petroleum jelly, cocoa butter, lanolin, and other emulsifying agents, salts for varying the osmotic pressure or buffers for securing an appropriate pH-value of the composition can be used as auxiliary agents.

Furthermore, the composition may contain other therapeutically active components which may appropriately be administered together with the compounds of the invention in the treatment of infectious diseases such as other suitable antibiotics, in particular such antibiotics which may enhance the activity and/or prevent development of resistance. Such antibiotics include penicillins, cephalosporins, tetracyclines, rifamycins, erythromycins, lincomycin, clindamycin and fluoroquinolones. Other compounds which advantageously may be combined with the compounds of the invention, especially in topical preparations, include e.g. corticosteroids, such as hydrocortisone or triamcinolone. Alternatively, such other therapeutically active component(s) may be administered concomitantly (either simultaneously or sequentially) with the composition of the invention.

For granulates, tablets, capsules or dragees the pharmaceutical composition of the invention appropriately contains from 25% to 98% of the active ingredient of the invention, and in oral suspensions the corresponding amount is appropriately from 2% to 20% active ingredient.

When the active ingredient is administered in the form of salts with pharmaceutically acceptable non-toxic acids or bases, preferred salts are for instance easily water-soluble or sparingly soluble in water, in order to obtain a particular and appropriate rate of absorption.

As indicated above, the compounds of formula I and their salts may be included in pharmaceutical formulations, including suspensions, ointments and creams. A pharmaceutical preparation for oral administration may also be in form of a suspension of the active ingredient as such or in the form of a sparingly water-soluble pharmaceutically acceptable salt, the preparation containing from 20 to 100 mg per ml of vehicle. A pharmaceutical preparation for topical treatment may be in the form of an ointment or cream containing the active ingredient in an amount of from 0.5 to 50% of preparation. Topical preparations are favourable due to the stability towards sunlight and the relatively lipophilic nature of the present compounds.

The dose of the compounds of the invention may suitably be selected so that the desired activity may be achieved without serious adverse effects. In the human systemic therapy the compounds and their salts are conveniently administered (to adults) in dosage units containing no less than 50 mg and up to 1000 mg, preferably from 200 to 750 mg, calculated as the compound of formula I.

By the term “dosage unit” is meant a unitary, i.e. a single, dose which is capable of being administered to a patient, and which may be readily handled and packed, remaining as a physically and chemically stable unit dose comprising either the active ingredient alone or in admixture with one or more solid or liquid pharmaceutical diluents or vehicles.

In the form of a dosage unit, the compound may be administered one or more times a day at appropriate intervals, always depending, however, on the condition of the patient, and in accordance with the prescription made by the medical practitioner.

Thus in systemic treatment a daily dosage will preferably be an amount of from 0.5 to 3 g of the active ingredient.

The term “usage unit” in connection with topical use means a unitary, i.e. a single dose capable of being administered topically to a patient in an application per square centimetre of the infected area of from 0.1 mg to 10 mg and preferably from 0.2 mg to 1 mg of the active ingredient in question.

If the composition is to be injected, a sealed ampoule, a vial or a similar container may be provided containing a parenterally acceptable sterile aqueous or oily injectable solution or dispersion of the active ingredient as the dosage unit.

The parenteral preparations are in particular useful in the treatment of conditions in which a quick response to the treatment is desirable. In the continuous therapy of patients suffering from infectious diseases, the tablets or capsules may be the appropriate form of pharmaceutical preparation owing to the prolonged effect obtained when the drug is given orally, in particular in the form of sustained-release tablets.

In the treatment of infectious diseases, such tablets may advantageously contain other active components as mentioned above.

In the method of treating patients suffering from infectious disease, the compound of formula I or an equivalent amount of a salt or ester thereof may suitably be administered to patients in a dose of from 0.03 g to 0.7 g/kg body weight per day in 1 to 3 doses, preferably from 0.5 g to 3 g per day. Preferably, the active ingredient is administered in the form of dosage units as indicated above.

Patients that may receive a treatment or be administered a treatment of the present invention include animals, including mammals, and particularly humans. Animals also include domestic animals, such as horses, cows, pigs, sheep, poultry, fish, cats, dogs and zoo animals.

The treatment of infectious diseases may often involve determining whether said disease is resistant or refractory to the treatment before the treatment is, in fact, initiated. By way of example, samples containing the infectious microbe may be taken from the patient, e.g. blood or urine, whereafter the sample is cultured and exposed to the treatment to see whether said infectious organism responds to the treatment. Accordingly, the present invention also provides a method for identifying compounds with antimicrobial effect comprising contacting a microorganism with a compound of formula I, optionally together with other therapeutically active agents, and determining whether said compound or mixture of compounds has a toxic or static effect on the microorganism in question.

The compositions of the present invention are not limited to pharmaceuticals, but may also be used in a non-therapeutic context to control microbial growth. By way of example, the selectivity of antimicrobial agents render them useful to enhance growth of particular microorganisms(s) (such as non-pathogenic microorganisms) at the expense of others in a multi-species culture.

The invention is further described in the following Preparations and Examples which are not in any way intended to limit the scope of the invention as claimed.

### PREPARATIONS AND EXAMPLES

Methods for Preparing Compounds of the Invention

Steroid Starting Materials

The starting carboxylic acid substituted steroid analogues may be obtained commercially or prepared by methods described in the literature. Steroids related to fusidic acid may be prepared according to various literature procedures starting from natural fusidanes such as fusidic acid, helvolic acid, viridominic acids and compounds from the cephalosporin P family (see e.g. Godtfredsen and Vangedal, 1962; Arigoni et al., 1964; Godtfredsen et al., 1965ₐ and 1965_{b}; Godtfredsen et al., 1966; Diassi et al., 1966; von Daehne et al 1979, and references cited therein, the disclosures of which are incorporated herein by reference) or by simple chemical modifications of the above-mentioned fusidanes including hydrogenation of double bonds, dehydration reactions, sulfation and oxidation, well known to those skilled in the art.

Sulfation of Hydroxy Groups:

All compounds of the invention containing one or several free hydroxy groups may be sulfated either selectively at one hydroxy group or at several hydroxy group using stecheometric or excess amounts of sulfur trioxide-pyridine complex, respectively as reported in the litterature (Kinney et al., 2000). Sulfatation is carried out prior to coupling reactions A, B and C.

Acylation of Hydroxy Groups

Acylation of the free hydroxy groups of steroid derivatives is carried out using an excess of acetic acid anhydride in pyridine at room temperature under anhydrous conditions.

Reduction of Double Bonds

Double bonds of steroid derivatives are carried out by means of catalytic hydrogenation using palladium on carbon as catalyst and acetic acid, MeOH, EtOH or ethyl acetate as solvent. The reactions are shaken for 6-20 h at room temperature.

Dehydration of Hydroxy Groups

Dehydration of 11-OH of fusidic acid derivatives is achieved by treating fusidic acid derivatives by excess thionyl chloride in pyridine and dichloromethan at 0° C. under anhydrous conditions.

Removal of the 16-acetoxy Group

The 16-acetoxy group of fusidic acid derivatives can be removed by reacting the corresponding methyl ester in refluxing anhydrous methanol in presence of excess magnesium turnings under anhydrous conditions. The methyl ester is then removed by refluxing in aqueous sodium hydroxide for 1 h.

Oxidation of Hydroxy Groups

Steroids containing keto or aldehyde functionalities can be obtained from the corresponding alcohols by various oxidation methods well known to those skilled in the art.

Branched Polyamine Starting Materials

Branched polyamines are generally chosen from those commercially available, e.g. those found in the Available Chemicals Directory (ACD) database, but can also be synthesized by methods known from the literature (selected references: Goodnow et al., 1990; Bergeron et al., 1994; Stromgaard et al., 1999; Gaell and Blagbrough, 2000; Kuksa et al., 2000 and references cited therein; Karigiannis and Papaioannou, 2000 and references cited therein, the disclosures of which are incorporated herein by reference).

Synthesis of Steroids with a Branched Polyamine Moiety Linked Via an AMIDE BOND (METHOD A, Scheme 1)

Compounds of the invention where the branched polyamine moiety is linked to the steroid nucleus via an amide bond may be prepared from various steroids containing a carboxylic acid, e.g. from tetrahydrofusidic acid in scheme 1, and numerous branched polyamine compounds. The carboxylic acid group of a steroid derivative is esterified to produce a reactive ester, for example a succinimide ester, by reacting the carboxylic acid group with N-hydroxysuccinimide in anhydrous THF in presence of dicyclocarbodiimide (DCC). The succinimide ester may then be reacted with a branched polyamine by dissolving an excess of the branched polyamine in anhydrous chloroform under argon and then slowly adding a chloroform solution containing the activated ester. The reactions are performed at room temperature and are completed in between 6 and 24 hours. After this time the reaction mixture can be concentrated without additional aqueous work-up procedures and directly purified by reversed phase HPLC using mixtures of acetonitrile and water buffered with trifluoroacetic acid as eluent or column cromatography on silica gel using mixtures of dichloromethan, methanol and aqueous amonia as eluent. The method is illustrated by an example in Scheme 1, where the steroid nucleus is represented by tetrahydrofusidic acid. Tetrahydrofusidic acid is first converted to the corresponding N-succinimide ester by reaction with N-hydroxysuccinimide in anhydrous THF in presence of dicyclocarbodiimide. Said tetrahydofusidic acid ester is then reacted with N,N-bis(2-aminoethyl)ethane-1,2-diamine by dissolving an excess (3 equivalents) N,N-bis(2-aminoethyl)ethane-1,2-diamine in anhydrous chloroform under argon and then slowly (over 30 min) adding a chloroform solution containing the activated ester. Solvents are evaporated under reduced pressure and the resulting crude oil is purified on silica gel using a mixture of dichloromethan, methanol and 25% aqueous ammonia as eluent. A white powder is obtained after freeze drying of purified product, Compound 101.
Method A 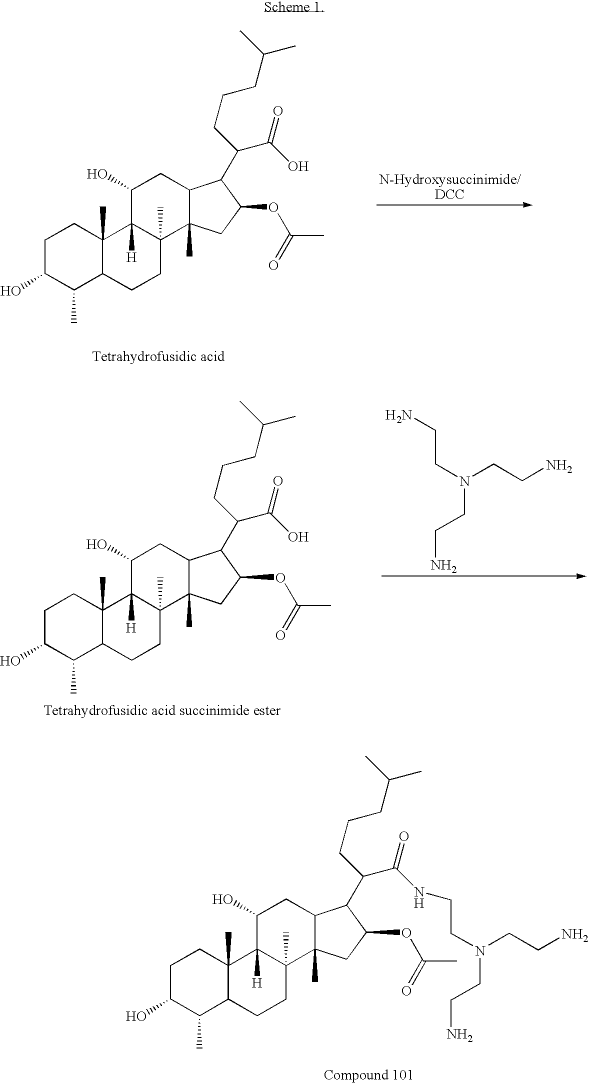
Synthesis of Steroids with a Branched Polyamine Moiety Linked Via an Amide Bond (Method B, Scheme 2)

Alternatively, the compounds of the invention with the formula V can be prepared by reacting anhydrides of the steroid acid, e.g. fusidic acid anhydride in scheme 2, with excess of the branched polyamine, e.g. N,N-bis(2-aminoethyl)ethane-1,2-diamine, using the same reaction conditions described for method A, using a succinimide ester as starting material (Scheme 2). 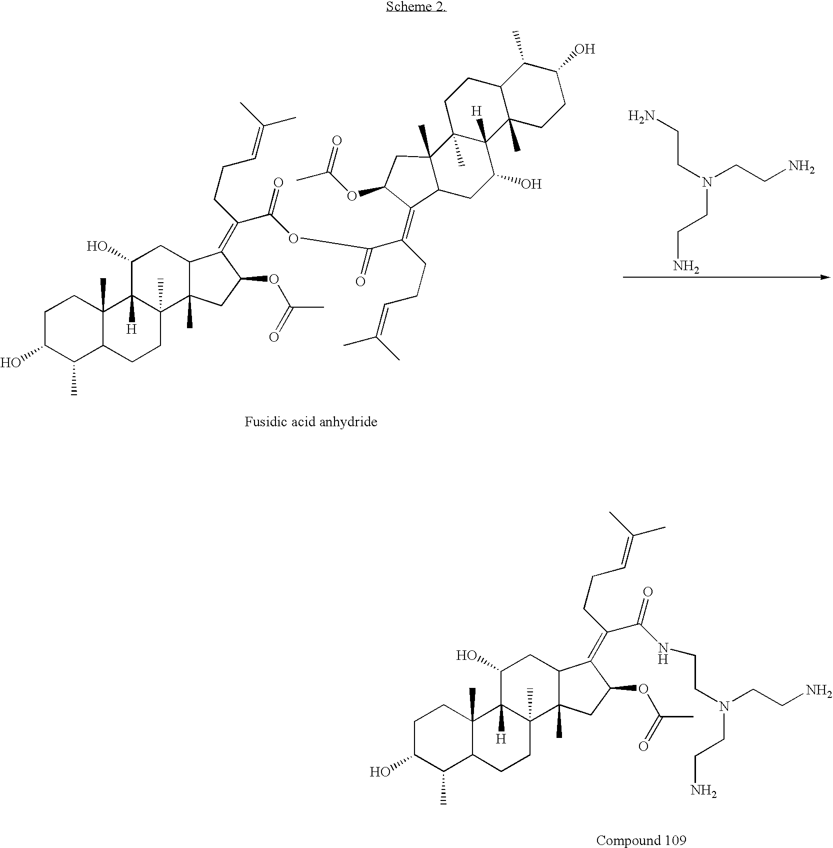
Reduction of Amide Bonds

The amide bonding resulting from the reaction of a branched polyamine and a succinimide ester or carboxylic acid anhydride described in scheme 1 and 2, respectively (e.g. compounds of formula IV and V) can be reduced to the corresponding amine by reacting the amide with a 10 fold excess of diborane in refluxing THF for 5-10 hours. The reaction mixture is then acidified with 4N aqueous hydrochloric acid to pH 1 and stirred vigorously for 2-4 hours. The reaction mixture is then freeze dried and the resulting white powder is purified on silica gel using a mixture of dichloromethan, methanol and 25% aqueous ammonia as eluant. A white powder is obtained after freeze drying of purified product.

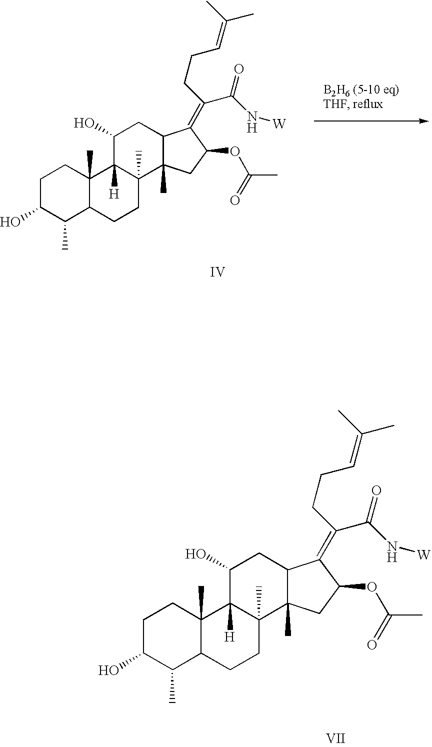

Scheme 3. Preparation of C-21 polyaminated fusidic acid analogues of formula IV, wherein W represents a radical of the formula -(Z)ₙ-(NR-Z)ₚ-NR₂.

Introduction of Branched Polyamines by Reductive Amination of Ketones (Method C, Scheme 4)

Compounds of the invention where the branched polyamine moiety is linked to various sites of the steroid nucleus can be prepared from steroid analogues containing a keto or aldehyde functionality where substitution with the branched polyamine is desired. The appropriate steroid can be obtained from commercial sources or can be synthesized by various methods known to those skilled in the art (e.g. various oxidation methods, reduction of carboxylic esters, etc.). The carbonyl functionalized steroid can be reacted directly with the unprotected polyamine building block by means of reductive amination using methods reported for the preparation of synthetic squalamines (Pechulis et al., 1995; Weis et al., 1999; Kinney et al., 2000). Alternatively, an steroid containing an amino group can then be reacted with appropriate Boc-protected polyamine fragments containing an aldehyde function by means of reductive amination as described in the literature for the preparation squalamine equivalents substituted at C-3 with a spermidine chain (Hon-Seok Kim et al., 2000). Finally, the Boc-protective groups can be cleaved with trifluoroacetic acid and purified as described above.

The method is illustrated by an example in Scheme 4 where the fusidic acid nucleus is represented by 3-keto fusidic acid. To a solution of 3-keto-fusidic acid (1 equivalent) in methanol was added successively N,N-bis(2-aminoethyl)ethane-1,2-diamine (3 equivalents), acetic acid and NaBH(OAc)₃ (3 equivalents) and the resulting reaction mixture was stirred for 6-16 h after which time methanol is evaporated under reduced pressure resulting a pale yellow oil. Pure Compound 118 is obtained after chromatography on silica gel using a mixture of dichloromethan, methanol and 25% aqueous ammonia as eluant. A white powder of pure Compound 118 is obtained after freeze drying of purified product in yields ranging from 70-85%.
Method C 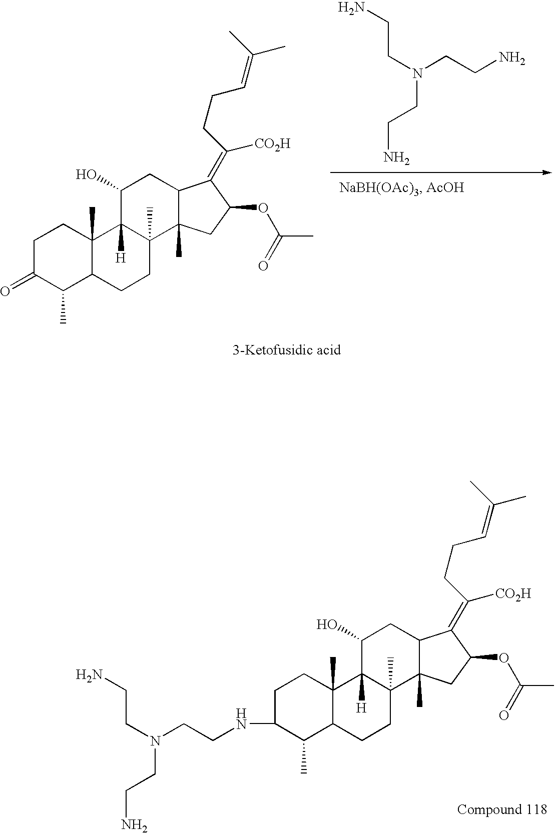
Scheme 4. Representative example for the introduction of branched polyamine fragments to a steriod nucleus containing a carbonyl function via reductive amination using NaBH(OAc)₃as reducing agent (Abdel-Magid, 1996).
Purification of the Compounds of the Invention:

The resulting compounds of the invention can be purified by column chromatography on silica gel 60 (E. Merck), 230-400 mesh using mixtures of dichloromethan, methanol and aqueous ammonia as eluent. Alternatively, the compounds of the invention can be purified by reversed phase preparative high performance liquid chromatography (HPLC) using acetonitrile buffered with trifluoroacetic acid or acetic acid as eluent.

Examples of the invention prepared according to general methods A, B and C:

| | | | |
|---|---|---|---|
| | | Steroid | Branched |
| Comp. | | starting | polyamine |
| no | Method | material | starting material |

| | | | |
|---|---|---|---|
| 101 | A | Tetrahydrofusidic | N,N-bis(2- |
| | | acid-N-succinimide | aminoethyl) |
| | | ester | ethane-1,2-diamine |

¹³C NMR (CD₃OD), δ/ppm: 177.5, 172.6, 80.2, 72.4, 68.9, 57.6, 54.9, 51.3, 50.3, 50.2,
41.5, 41.4, 41.1, 40.1, 38.5, 38.3, 38.0, 37.1, 36.4, 33.1, 31.7, 31.1, 29.1, 26.4,
23.9, 23.3, 23.1, 23.0, 22.6, 21.4, 17.1, 16.5

| | | | |
|---|---|---|---|
| 102 | A | 11-Desoxy- | N,N-bis(2- |
| | | tetrahydrofusidic | aminoethyl) |
| | | acid-N-succinimide | ethane-1,2-diamine |
| | | ester | |

¹³C NMR (CD₃OD), δ/ppm: 177.4, 172.6, 80.4, 72.5, 57.7, 55.0, 51.7, 50.9, 50.5, 46.4
45.9, 41.0, 40.4, 40.2, 40.1, 39.0, 38.5, 37.4, 36.4, 34.5, 32.0, 31.1, 29.9, 29.1, 26.8,
26.4, 24.4, 23.1, 22.9, 21.6, 21.4, 21.3, 20.7, 17.8, 16.5

| | | | |
|---|---|---|---|
| 103 | A | 16-Desacetoxy | N,N-bis(2- |
| | | tetrahydrofusidic | aminoethyl) |
| | | acid-N-succinimide | ethane-1,2-diamine |
| | | ester | |

¹³C NMR (CD₃OD), δ/ppm: 178.6, 72.5, 69.3, 57.2, 54.7, 53.9, 51.8, 51.7, 43.8, 42.4, 41.5,
40.2, 40.0, 38.3, 38.3, 38.0, 37.1, 36.8,33.1, 32.6, 31.7, 31.2, 31.1, 29.1, 28.7, 26.5,
23.8, 23.2, 23.2, 22.9, 22.7, 16.6, 16.4

| | | | |
|---|---|---|---|
| 104 | A | 13(17)-en-16- | N,N-bis(2- |
| | | desacetoxytetrahydro | aminoethyl) |
| | | fusidic acid-N- | ethane-1,2-diamine |
| | | succinimide ester | |

¹³C NMR (CD₃OD), δ/ppm: 175.9, 143.4, 133.9, 72.5, 70.2, 58.6, 57.6, 56.1, 52.8, 45.3,
44.3, 40.0, 39.9, 39.1, 38.6, 37.9, 36.4, 35.6, 31.9, 31.8, 30.9, 30.7, 30.6, 29.3, 29.0
25.9, 24.9, 24.1,23.2, 23.1, 22.9, 22.9, 16.5

| | | | |
|---|---|---|---|
| 105 | A | 3β-tetrahydrofusidic | N,N-bis(2- |
| | | acid-N-succinimide | aminoethyl) |
| | | ester | ethane-1,2-diamine |

¹³C NMR (CD₃OD), δ/ppm: 177.5, 172.6, 80.2, 77.3, 68.8, 57.1, 54.8, 51.3, 51.0, 50.3
50.2, 44.3, 41.5, 41.3, 41.2, 41.1, 40.1, 40.0, 38.4, 37.8, 36.6, 35.3, 33.7, 32.7, 31.7,
29.1, 26.4, 24.5, 23.6, 23.2, 23.0, 22.7, 21.4, 17.1, 16.0

| | | | |
|---|---|---|---|
| 106 | A | 9(11)-en- | N,N-bis(2- |
| | | tetrahydrofusidic | aminoethyl) |
| | | acid-N-succinimide | ethane-1,2-diamine |
| | | ester | |

¹³C NMR (CD₃OD), δ/ppm: 177.2, 172.6, 153.4, 118.6, 81.2, 71.9, 57.5, 55.0, 52.1, 42.9,
42.4, 40.7, 40.0, 39.3, 39.1, 38.5, 35.1, 34.4, 32.5, 30.8, 29.9, 29.1, 26.4, 26.3, 24.5,
23.1, 22.9, 22.5, 22.4, 21.3, 18.2, 18.2, 16.1

| | | | |
|---|---|---|---|
| 107 | A | deoxycholic acid-N- | N,N-bis(2- |
| | | succinimide ester | aminoethyl) |
| | | | ethane-1,2-diamine |

¹³C NMR (CD₃OD), δ/ppm: 177.0, 74.0, 72.6, 57.6, 57.1, 55.2, 48.1, 47.6, 43.7, 40.5, 40.0,
38.7, 37.5, 37.3, 36.9, 36.5, 35.3, 34.9, 34.2, 33.4, 31.1, 30.0, 28.7, 28.4, 27.5, 24.9,
23.7, 17.7, 13.2

| | | | |
|---|---|---|---|
| 108 | A | 23,24-bisnor-5- | N,N-bis(2- |
| | | cholenic acid-3β-ol- | aminoethyl) |
| | | N-succinimide ester | ethane-1,2-diamine |

¹³C NMR (CD₃OD), δ/ppm: 179.8, 142.3, 122.4, 72.4, 57.9, 57.7, 57.4, 55.1, 54.1, 51.7,
45.1, 43.5, 43.1, 41.0, 40.1, 38.6, 38.3, 37.7, 33.3, 33.0, 32.3, 28.5, 25.4, 22.2, 19.9,
18.0, 12.6

| | | | |
|---|---|---|---|
| 109 | B | Fusidic acid | N,N-bis(2- |
| | | anhydride | aminoethyl) |
| | | | ethane-1,2-diamine |

¹³C NMR (CD₃OD), δ/ppm: 171.4, 171.1, 140.8, 135.6, 132.1, 123.5, 73.6, 71.4, 68.3, 56.5,
53.1, 49.3, 48.7, 43.1, 39.6, 39.5, 39.3, 37.7, 37.1, 36.3, 36.2, 35.6, 32.4, 30.3, 30.0,
29.4, 28.0, 25.7, 24.2, 22.8, 21.1, 20.8, 17.9, 17.6, 16.0

| | | | |
|---|---|---|---|
| 110 | B | Fusidic acid | N,N-bis(3- |
| | | anhydride | aminopropyl) |
| | | | propane-1,3- |
| | | | diamine |

¹³C NMR (CD₃OD), δ/ppm: 174.3, 172.4, 143.4, 135.8, 133.3, 124.6, 75.3, 72.5, 68.6, 52.8,
50.7, 44.6, 40/9, 40.7, 40.3, 39.1, 38.2, 37.9, 37.4, 36.9, 32.9, 31.1, 31.0, 30.5, 29.7,
28.8, 27.5, 25.9, 23.9, 23.8, 22.4, 21.3, 18.0, 17.9, 16.5

| | | | |
|---|---|---|---|
| 111 | A | 11-desoxy-3- | N,N-bis(2- |
| | | OXO₃H- | aminoethyl) |
| | | tetrahydrofusidic | ethane-1,2-diamine |
| | | acid-N-succinimide | |
| | | ester | |

¹³C NMR (CD₃OD), δ/ppm: 177.6, 172.6, 81.6, 80.7, 54.8, 52.0, 50.9, 50.6, 46.5, 46.0,
41.0, 40.5, 40.1, 39.7, 39.4, 39.4, 38.2, 37.1, 36.1, 34.1, 32.3, 30.3, 29.1, 28.4, 26.9, 26.4
24.5, 23.1, 23.0, 21.7, 21.4, 21.2, 21.0, 17.89, 16.4

| | | | |
|---|---|---|---|
| 112 | A | 11-desoxy-16- | N,N-bis(2- |
| | | desacetoxy- | aminoethyl) |
| | | 17S,20,24,25- | ethane-1,2-diamine |
| | | tetrahydrofusidic | |
| | | acid-N-succinimide | |
| | | ester | |

¹³C NMR (CD₃OD), δ/ppm: 178.6, 72.5, 57.4, 55.0, 54.3, 52.0, 47.4, 46.4, 44.5, 40.6, 40.1,
40.1, 39.0, 38.4, 37.5, 36.3, 34.6, 32.7, 31.7, 31.1, 30.0, 29.1, 28.7, 27.3,26.5, 24.7
23.2, 22.9, 21/5, 21.4, 20.7, 17.2, 16.6

| | | | |
|---|---|---|---|
| 113 | A | Tetrahydrofusidic | N,N-bis(3- |
| | | acid-N-succinimide | aminopropyl) |
| | | ester | propane-1,3- |
| | | | diamine |

¹³C NMR (CD₃OD), δ/ppm: 177.4, 172.6, 80.1, 72.5, 68.9, 53.0, 52.9, 51.5, 51.3, 50.3,
50.1, 41.5, 41.4, 41.1, 41.0, 38.9, 38.3, 37.9, 37.0, 36.4, 33.1, 31.8, 31.1, 30.0,
29.2, 27.7, 26.4, 23.9, 23.3, 23.2, 23.0, 22.6, 21.4, 17.1, 16.5

| | | | |
|---|---|---|---|
| 114 | A | 23,24-bisnor-5- | N,N-bis(3- |
| | | cholenic acid-3β-ol- | aminopropyl) |
| | | N-succinimide ester | propane-1,3- |
| | | | diamine |

¹³C NMR (CD₃OD), δ/ppm: 179.6, 142.3, 122.3, 72.4, 57.9, 54.1, 52.9, 52.7, 45.1,
43.5,43.0, 41.0, 40.9, 38.6, 37.7, 33.3, 33.0, 32.3, 29.8, 28.5, 27.7, 25.4, 22.2, 19.9, 18.0, 12.5

| | | | |
|---|---|---|---|
| 115 | A | 3-OAc- | N,N-bis(3- |
| | | tetrahydrofusidic | aminopropyl) |
| | | acid-N-succinimide | propane-1,3- |
| | | | diamine |
| | | | ester |

¹³C NMR (CD₃OD), δ/ppm: 177.3, 172.9, 172.6, 80.1 76.2, 68.6, 52.9, 51.4, 51.1, 50.3,
50.2, 41.5, 41.3, 41.1, 40.9, 40.1, 38.9, 38.0, 36.6, 33.5, 31.7, 30.0, 29.1, 28.3, 27.7, 26.4, 23.7,
23.4, 23.2,23.0, 22.3, 21.4, 21.2, 17.2, 16.1

| | | | |
|---|---|---|---|
| 116 | A | 3-OSO₃ | N,N-bis(3- |
| | | H-11- | aminopropyl) |
| | | desoxy- | propane-1,3- |
| | | tetrahydrofusidic | diamine |
| | | acid N-succinimide | |
| | | ester | |

¹³C NMR (CD₃OD), δ/ppm: 177.1, 172.6, 81.7, 81.4, 52.9, 52.5, 52.3, 51.1, 50.7, 46.5,
46.2, 40.9, 40.6, 40.3, 40.0, 39.3, 38.7, 38.6, 37.0, 36.2, 33.5, 32.7, 30.5, 29.1, 28.3,
27.8, 27.5, 27.0, 26.4, 24.8, 23.1, 22.9, 21.6, 21.3, 21.3, 21.2, 17.9, 16.4

| | | | |
|---|---|---|---|
| 117 | A | 11-desoxy-16- | N,N-bis(3- |
| | | desacetoxy- | aminopropyl |
| | | 175,20,24,25- | propane-1,3- |
| | | tetrahydrofusidic | diamine |
| | | acid N-succinimide | |
| | | ester | |

¹³C NMR (CD₃OD), δ/ppm: 178.4, 72.5, 54.5, 52.9, 52.0, 47.4, 46.4, 44.5,41.0, 40.6, 40.1,
38.9, 38.8, 37.5, 36.3, 34.6, 32.9, 31.7, 31.2, 30.2, 30.1, 29.1, 28.8, 27.7, 27.3, 26.5,
24.7, 23.2,22.9,21.5, 21.4, 20.7, 17.2, 16.6

| | | | |
|---|---|---|---|
| 118 | C | 3-ketofusic | N,N-bis(2- |
| | | acid | aminoethyl) |
| | | | ethane-1,2-diamine |

¹³C NMR (CD₃OD), δ/ppm: 178.8, 173.2, 139.4, 138.5, 132.6, 125.3, 75.8, 69.0, 61.8, 52.6
51.6, 50.0, 50.7, 45.9, 43.9, 40.9, 40.2, 38.2, 37.5, 37.4, 37.0, 31.3, 30.9, 30.5, 29.3,
25.9, 24.9, 24.8, 23.3, 22.8, 21.1,18.0, 17.5, 16.0

| | | | |
|---|---|---|---|
| 119 | A | Tetrahydrofusidic | N-(3- |
| | | acid N-succinimide | aminopropyl)-N- |
| | | ester | methylpropane- |
| | | | 1,3-diamine |

¹³C NMR (CD₃OD), δ/ppm: 177.4, 172.6, 80.2, 72.5, 68.9, 56.6, 56.5, 51.6, 51.3, 50.4,
50.2, 42.3, 41.6, 41.4, 41.1, 40.9, 40.1 38.7, 38.3, 38.0, 37.1, 36.4, 33.1, 31.8, 31.1,
29.9, 29.2, 27.8, 26.4, 23.9, 23.3, 23.2, 23.0, 22.6, 21.4, 17.1, 16.5

| | | | |
|---|---|---|---|
| 120 | A | 11-desoxtertrahydro | N-(3- |
| | | fusidic acid N- | aminopropyl)-N- |
| | | succinimide ester | methylpropane- |
| | | | 1,3-diamine |

¹³C NMR (CD₃OD), δ/ppm: 177.4, 172.6, 80.5, 72.4, 56.6, 56.6, 52.0, 50.9, 50.5, 46.6,
45.9, 42.3, 41.0, 40.4, 40.0, 39.0, 38.7, 37.4, 36.3, 34.5, 32.1, 31.1, 30.3, 30.0, 29.1
27.7, 26.8, 26.4, 24.5, 23.1, 22.9, 21.6, 21.3, 21.3, 20.7, 17.8, 16.5

| | | | |
|---|---|---|---|
| 121 | A | 16-desacetoxy- | N-(3- |
| | | 17S20,24,25- | aminopropyl)-N- |
| | | tetrahydrofusidic | methylpropane- |
| | | acid N-succinimide | 1,3-diamine |
| | | ester | |

¹³C NMR (CD₃OD), δ/ppm: 178.5, 72.5, 69.2, 56.6, 54.1, 51.8, 51.6, 43.8, 42.4, 42.3,
41.5, 40.9, 40.1 38.8, 38.3, 37.9, 37.1, 36.8, 33.1,32.6, 31.6, 31.1, 31.1, 30.0, 29.1,
28.6, 27.7, 26.5, 23.8, 23.2, 22.9, 22.7, 16.6, 16.4

| | | | |
|---|---|---|---|
| 122 | A | deoxycholic acid N- | N-(3- |
| | | succinimide ester | aminopropyl)-N- |
| | | | methylpropane- |
| | | | 1,3-diamine |

¹³C NMR (CD₃OD), δ/ppm: 176.8, 74.0, 72.6, 56.5, 56.3, 47.6, 43.7, 42.3, 40.9, 38.7, 37.5,
37.2, 36.9, 36.5, 35.3, 34.9, 34.2, 33.4, 31.1, 30.0, 30.0, 28.7, 28.4, 27.8, 27.5, 24.9,
23.7, 17.7, 13.3

| | | | |
|---|---|---|---|
| 123 | A | 11-desoxy-16- | N-(3- |
| | | desacetoxy- | aminopropyl)-N- |
| | | 17S20,24,25- | methylpropane- |
| | | tetrahydrofusidic | 1,3-diamine |
| | | acid N-succinimide | |
| | | ester | |

¹³C NMR (CD₃OD), δ/ppm: 178.5, 72.5, 56.7, 56.6, 54.6, 52.0, 47.5, 46.4, 44.5, 42.3, 41.0,
40.6, 40.1, 39.0, 38.7, 37.5, 36.3, 34.6, 32.9, 31.7, 31.2, 30.0, 29.1, 28.9, 27.7, 27.3,
26.6, 24.7, 23.2, 22.9, 21.5, 21.4, 20.7, 17.2, 16.6

| | | | |
|---|---|---|---|
| 124 | C | 3-ketofusidic | N,N-bis(3- |
| | | acid | aminopropyl) |
| | | | propane-1,3- |
| | | | diamine |

¹³C NMR (CD₃OD), δ/ppm: 179.6, 173.3, 139,7, 137.8, 132.4, 1245.5, 75.9, 69.0, 60.8, 53.1,
52.5, 50.9, 50.0, 49.9, 47.9, 43.7, 40.8, 40.2, 37.6, 37.3, 31.8, 31.1, 30.6, 29.3, 27.7,
26.4, 25.9, 25.6, 24.8, 23.4, 22.7, 21.2, 18.0, 17.6, 16.5

| | | | |
|---|---|---|---|
| 125 | C | 3-ketofusidic | N-(3- |
| | | acid | aminopropyl)-N- |
| | | | methylpropane- |
| | | | 1,3-diamine |

¹³C NMR (CD₃OD), δ/ppm: 179.6, 173.3, 139.7, 137.5, 132.3, 125.6, 76.0, 69.0, 61.3, 57.3,
56.2, 50.8, 50.0, 43.6, 42.2, 40.8, 40.3, 37.5, 37.4, 37.0, 31.7, 31.1, 30.5, 29.2, 27.6, 25.9,
25.4, 25.0, 24.7, 23.3, 22.7, 21.2, 18.0, 17.6, 16.4

| | | | |
|---|---|---|---|
| 126 | A | Tetrahydrofusidic | N,N′-Bis-(3- |
| | | acid N-succinimide | amino-propyl)- |
| | | ester | N,N′-diamethyl- |
| | | | butane-1,4- |
| | | | diamine |
| 127 | A | Tetrahydrofusidic | N′1′-{3-[(3- |
| | | acid N-succinimide | Amino-propyl)- |
| | | ester | ethyl-amino]- |
| | | | propyl}-N′1′- |
| | | | ethyl-propane- |
| | | | 1,3- |
| | | | diamine |
| 128 | A | Tetrahydrofusidic | N,N′-Bis-(3- |
| | | acid N-succinimide | amino-propyl)- |
| | | ester | N,N′-diethyl- |
| | | | butane-1,4- |
| | | | diamine |
| 129 | A | 11-Desoxy- | N′1′-{3-[(3- |
| | | tetrahyfrofusidic | Amino-propyl)- |
| | | acid-N-succinimide | ethyl-amino]- |
| | | ester | propyl}-N′1′- |
| | | | ethyl-propane- |
| | | | 1,3-diamine |
| 130 | A | Tetrahydrofusidic | N,N′-Bis-(3- |
| | | acid N-succinimide | amino-propyl)- |
| | | ester | N,N′-bis- |
| | | | cyclopropylmethyl- |
| | | | butane-1,4- |
| | | | diamine |
| 131 | A | 11-Desoxy- | N′1′-(3-Amino- |
| | | tetrahyfrofusidic | propyl)-N′1′-(3- |
| | | acid-N-succinimide | dimethylamino- |
| | | ester | propyl)-propane- |
| | | | 1,3-diamine |

| Comp. | |
|---|---|
| no | Structure of compound |
| 101 | 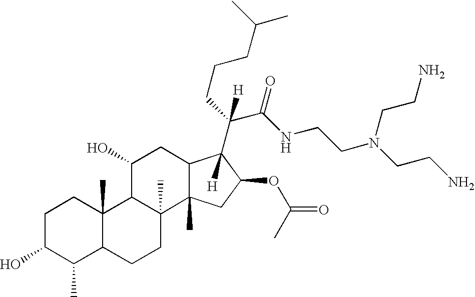 |
| 102 | 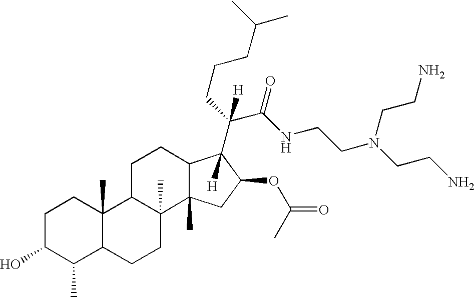 |
| 103 | 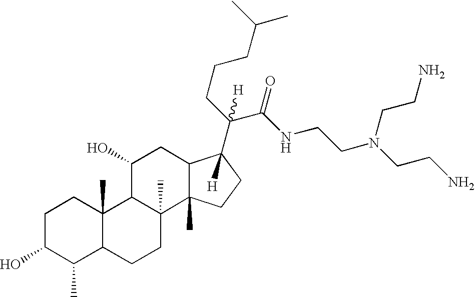 |
| 104 | 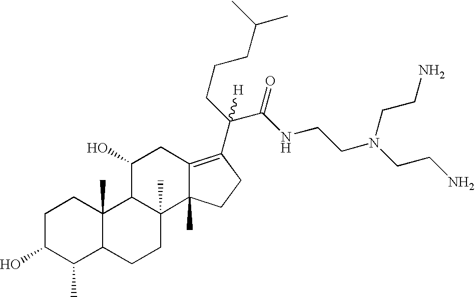 |
| 105 | 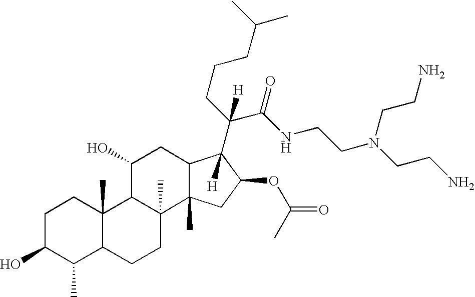 |
| 106 | 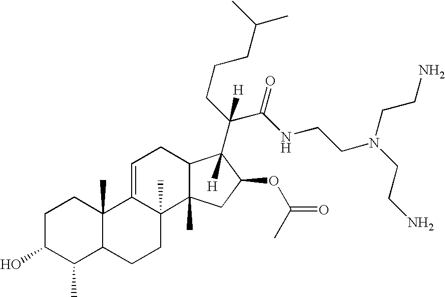 |
| 107 | 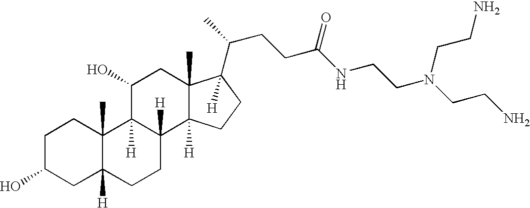 |
| 108 | 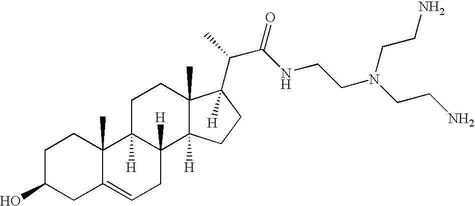 |
| 109 | 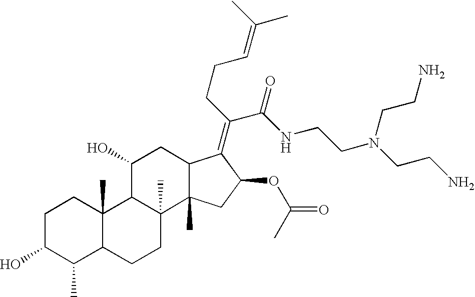 |
| 110 | 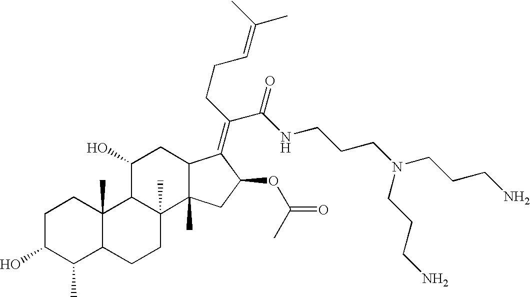 |
| 111 | 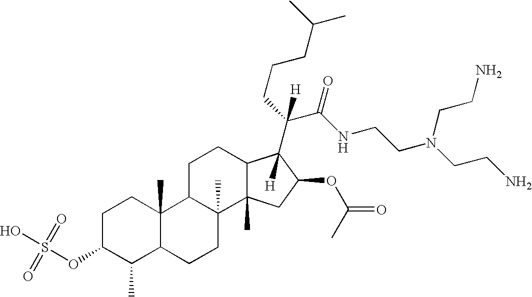 |
| 112 | 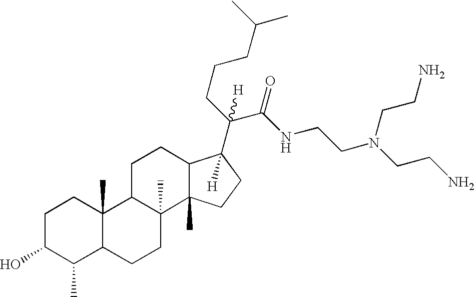 |
| 113 | 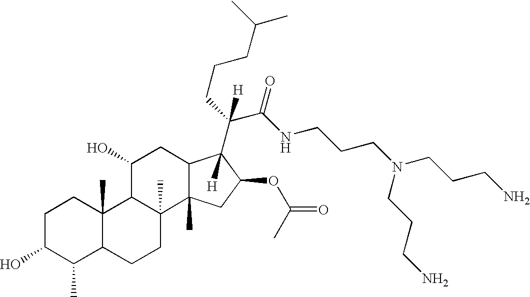 |
| 114 | 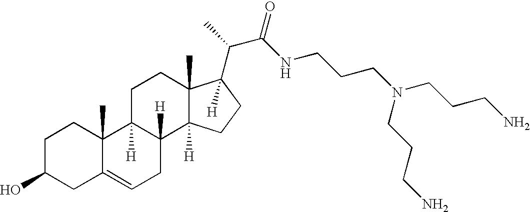 |
| 115 | 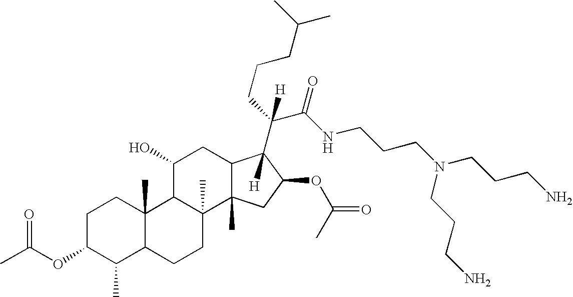 |
| 116 | 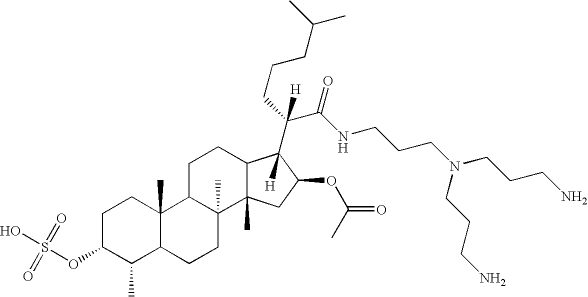 |
| 117 | 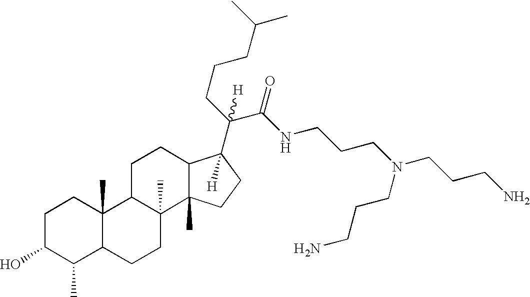 |
| 118 | 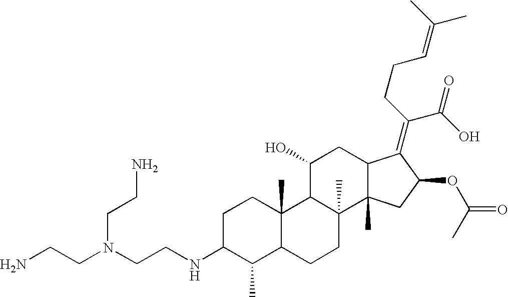 |
| 119 | 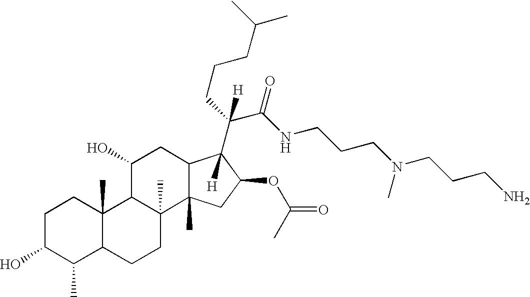 |
| 120 | 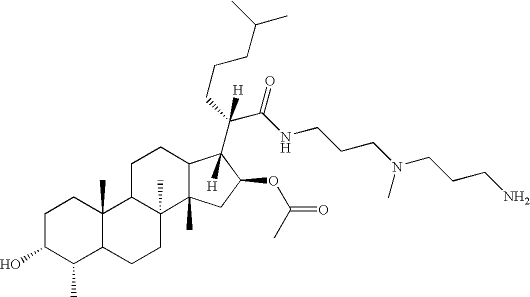 |
| 121 | 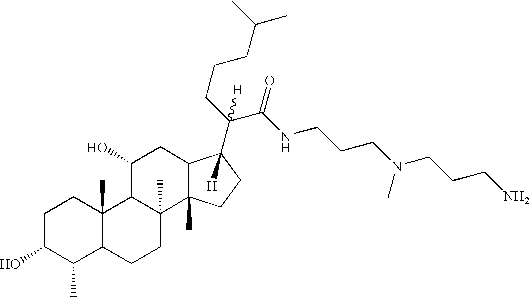 |
| 122 | 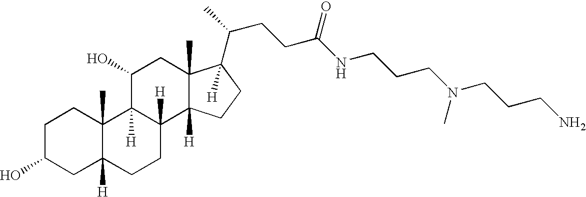 |
| 123 | 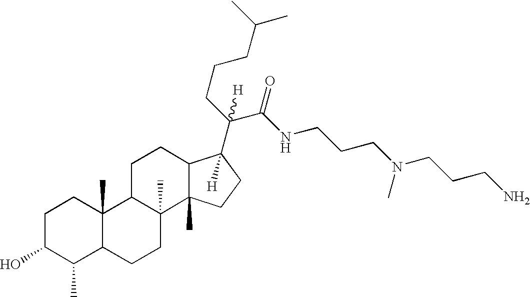 |
| 124 | 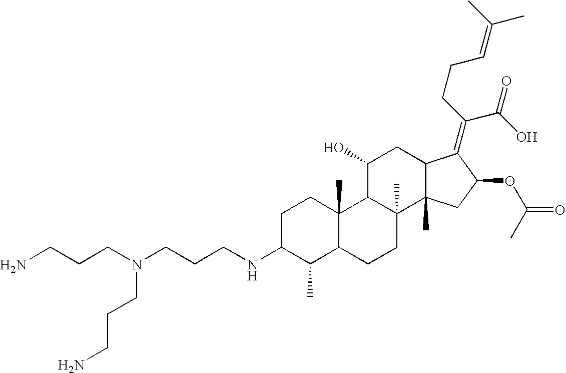 |
| 125 | 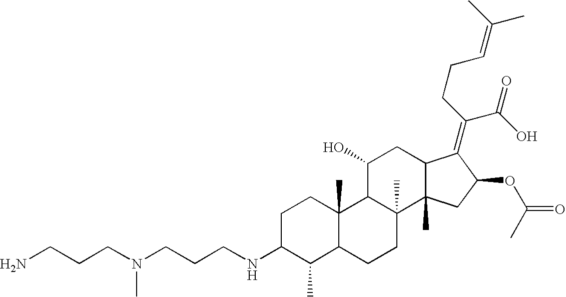 |
| 126 | 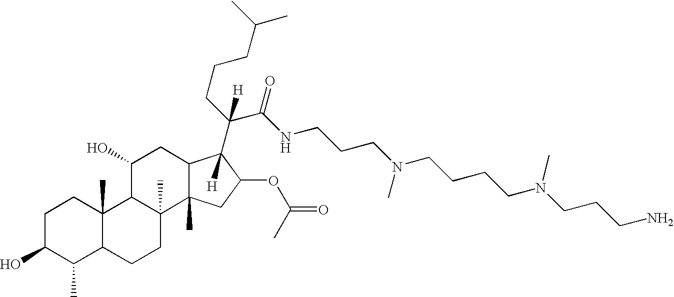 |
| 127 | 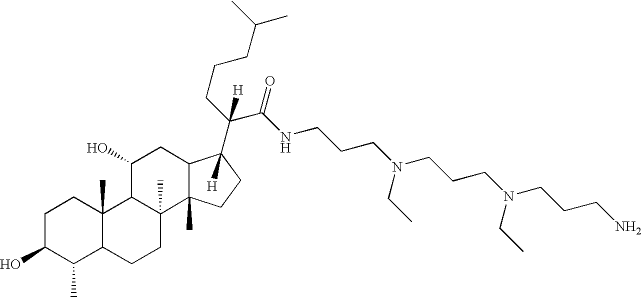 |
| 128 | 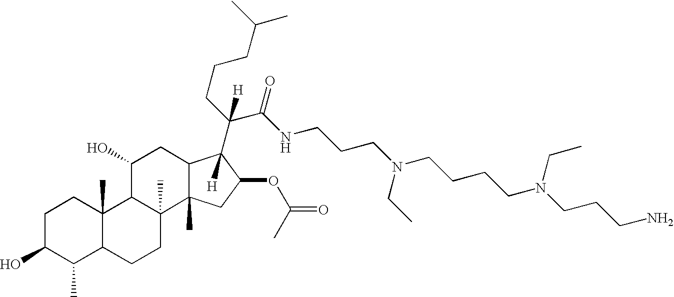 |
| 129 | 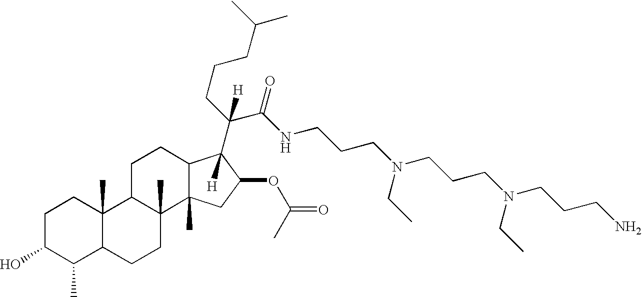 |
| 130 | 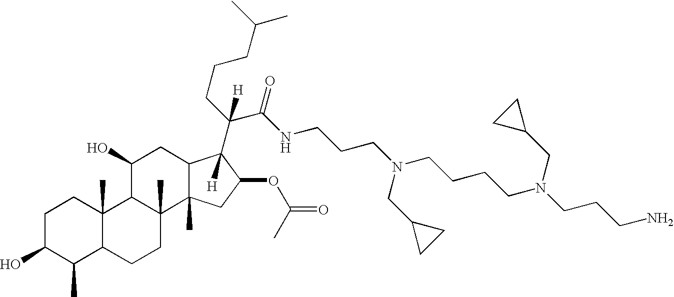 |

Antimicrobial Activity

In vitro investigations have shown a significant potency of the compounds of the invention against a large number of bacteria including gram-positive and gram-negative strains (Staphyiococci, Streptococci, Corynebacteriae, Mycobacteriae, Proteus, Propionibacterium, Pseudomonas, Neisseriae, E. coli) and fungal strains (Candida and Aspergillus). Biological tests have showed superior activity of compounds of the invention when compared to that reported for several natural squalamine analogues (WO 00/09137). The antibacterial activity of compounds of the invention is also comparable to that of related compounds reported in the literature (Moore et al, 1993; Kikuchi et al., 1997; Rao et al., 2000) and to known broad spectrum antibiotics such as ampicillin (Kikuchi et al., 1997). In addition, the studies of post-antibiotic effects point towards a strong bactericidal effect of the compounds of the invention. Table 1 shows MIC (Minimum Inhibitory Concentration) values of compounds of the invention towards a number of bacterial and fungal strains. Minimum Inhibitory Concentrations were estimated using an agar cup assay. Bacterial strains were obtained from the American Type Culture Collection or from our own collection of clinical isolates. Colonies from fresh overnight culture were resuspended in saline water to 0.5 MacFarland corresponding to 10⁸ CFU/ml. 200 ml Mueller Hinton agar (Oxoid) at 48° C. was inoculated at a concentration of 10⁶ CFU/ml and poured into square petri dishes (245×245 mm). Holes were made in the inoculated plates and 200 μl of the compounds to be tested were disposed into each hole. A dilution series of compounds contained six dilution between 0.25 and 125 μg/ml. For Streptococci Mueller Hinton agar was supplemented with 5% sheep blood. Plates were appropriately incubated and zone diameters of growth inhibition were measured using an electronic caliper. MICs were estimated using a linear regression curve between the zone diameter of growth inhibition and the log₂ of the sample concentration. The microbiological assay set up is in agreement with the European Pharmacopoeia 3rd edition (1997). The inhibition zones are function of the concentration of the compounds used. Known antibiotics including fusidic acid (FA), mupirocin and linezolid were used as reference compounds.

TABLE 1

| | Compounds of the invention and their in vitro activities MIC (mg/l) | | | | | | | | | | | | | | | |
|---|---|---|---|---|---|---|---|---|---|---|---|---|---|---|---|---|
| Microorganism/ | | | | | | | | | | | | | | | Line- | Mupi- |
| strain | 101 | 102 | 103 | 104 | 105 | 106 | 107 | 114 | 117 | 112 | 111 | 115 | 116 | FA | zolid | rocin |
| S. aureus | 4 | 4 | 1 | 16 | 16 | 4 | 16 | 16 | 1 | 1 | — | — | — | 0.02 | 1 | 0.5 |
| CJ247 | | | | | | | | | | | | | | | | |
| S. aureus | 4 | 4 | 1 | 16 | 16 | | 16 | 16 | 1 | 1 | 16 | 4 | 4 | 0.02 | 4 | 1 |
| CJ200 | | | | | | | | | | | | | | | | |
| S. aureus | 4 | 4 | 1 | 16 | 16 | 4 | 16 | 16 | 1 | 1 | 16 | 4 | 4 | 0.02 | 16 | 1 |
| CJ234R | | | | | | | | | | | | | | | | |
| S. aureus | 4 | 4 | 1 | 16 | 16 | 4 | 16 | 16 | 1 | 1 | 16 | — | 4 | 16 | 1 | 0.5 |
| CJ234F | | | | | | | | | | | | | | | | |
| S. aureus | 4 | 4 | 1 | 16 | 16 | 4 | 16 | 16 | 1 | 1 | — | — | — | 16 | — | — |
| N6 | | | | | | | | | | | | | | | | |
| S. epidermis | 4 | 4 | 1 | 16 | 16 | 4 | 16 | 16 | 1 | 1 | 4 | 4 | 4 | 0.02 | 0.25 | 0.04 |
| CK5 | | | | | | | | | | | | | | | | |
| Propionibacterium | 4 | 4 | 1 | 16 | 16 | 4 | 16 | 16 | — | — | 4 | 4 | 16 | 0.2 | 1 | — |
| acnes FN33 | | | | | | | | | | | | | | | | |
| Corynebacterium | 4 | 4 | 1 | 16 | 16 | 4 | 16 | 64 | 0.25 | 0.25 | 16 | 64 | 4 | 0.1 | — | — |
| xerosis FF | | | | | | | | | | | | | | | | |
| Streptococcus | 16 | 16 | 4 | 16 | 64 | 4 | 16 | 64 | — | — | 16 | 4 | 16 | 16 | 4 | 1 |
| pyogenes EC88 | | | | | | | | | | | | | | | | |
| Streptococcus | 16 | 16 | 16 | >64 | 64 | 16 | 16 | — | — | — | — | — | — | — | — | — |
| faecium EI19 | | | | | | | | | | | | | | | | |
| E. coli | 16 | 16 | 4 | >64 | 64 | 16 | 16 | 16 | — | — | 16 | 16 | 64 | >64 | — | — |
| HA165 | | | | | | | | | | | | | | | | |
| Pseudomonas | 64 | 16 | 16 | >64 | 16 | 16 | 16 | >125 | — | — | >125 | 16 | 125 | >64 | — | — |
| aeruginosa | | | | | | | | | | | | | | | | |
| BA17 | | | | | | | | | | | | | | | | |
| Saccaromyces | 4 | 16 | 4 | 16 | 64 | 16 | 4 | 64 | — | — | 64 | 16 | 16 | >64 | — | — |
| cervisiae ZZ7 | | | | | | | | | | | | | | | | |
| Candida albicans | 16 | 4 | 1 | 16 | 16 | 16 | 16 | >125 | — | — | 125 | 16 | 64 | >64 | — | — |
| ZA | | | | | | | | | | | | | | | | |
| Asbergillus niger | 4 | 4 | >125 | >64 | 64 | 63 | 16 | — | — | — | 64 | 125 | 64 | >64 | — | — |
| ZM35 | | | | | | | | | | | | | | | | |

Comment:
Very clear inhibition zones for all compounds listed in Table 1 indicate bactericidal action.
FA = fusidic acid
— = mising MIC value
Strains:
FF = Corynebacterium xerosis
EC88 = Streptococcus pyrogenes
CJ234(F) =Staphylococcus aureus (MRSA#, Fus. resistant)
CJ(N6) = Staphylococcus aureus (Fus. resistant)
CJ247 = Staphylococcus aureus
CJ234(R) = Staphylococcus aureus (MRSA#, Rifampicin resistant)
CJ1200 = Staphylococcus aureus
CK5 = Staphylococcus epidermidis
#MRSA: meticiline resistant S. aureus
BA17 = Pseudomonas
HJ = Proteus
EI119(P) = Streptococcus faecium (Penicillin resistant)
ZA = Candida albicans
HA165 = E. coli
ZZ7 = Saccharomyces cerevisiae
FN33 = Propionibacterium
ZM6 = Aspergillus flavus
ZM35 = Aspergillus niger

Minimum Bactericidal Concentration (MBC) for compound 102

10⁶ bacteria were inoculated in 3 ml growth media (S. aureus—LB broth, S. pyogenes—TH broth) containing approximately 2×MIC, 1×MIC, 0.5×MIC and O×MIC respectively of compound 102 (MIC being relative to the strain being tested). S. aureus strains were grown aerobically, and S. pyogenes anaerobically in a carbon dioxide enriched incubator. Samples were diluted and plated on LA-plates (S. aureus) or blood-agar plates (S. pyogenes) followed by 24 hours incubation at 37° C. before being scored for colonies.

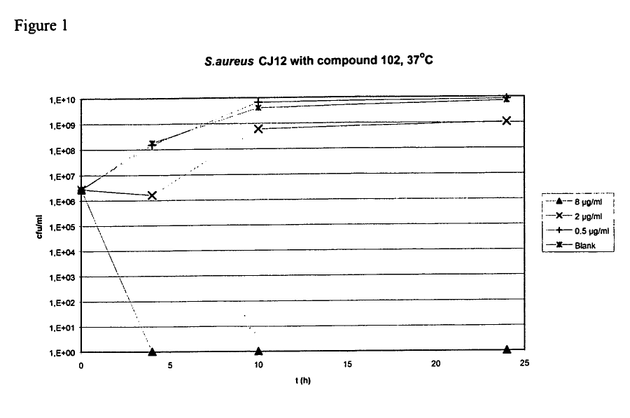

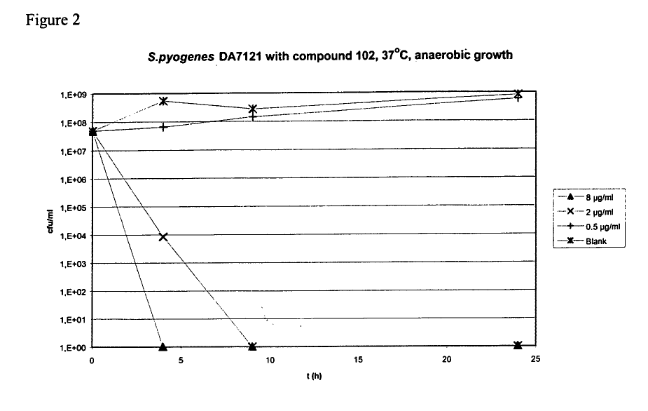

Compound 102 has a strong bactericidal impact on species of staphylococci and streptococci with strong bacterial killing at concentration twice that of MIC, as shown is FIGS. 1 and 2.

The data presented in Table 1 show that the compounds of the present invention generally exhibit a broad spectered activity towards the organisms tested. Moreover, they show activity towards strains which are resistant to standard antibiotics, such as fusidic acid, rifampicin and penicillin. The lack of cross-resistance lends support to the speculation that compounds of the present invention exert their anti-microbial activity through a mechanism which is different from known antibiotics. To overcome the increasing problem with resistance to antibiotics, it is vital to identify novel antibiotics with novel mechanisms of action.

### REFERENCES

Abedel-Magdid, A. F., Carcon, K. G., Harris, B. D., Maryanoff, C. A., Shah, R. D., J. Org. Chem., 1996, 3849-3862.
Arigoni, D., von Daehne, W., Godtfredsen, W. O., Malera, A., Vangedal, S., 1964, Experimentia, 1-4.
Bergeron, R. J., McManis, J. S., Liu, C. Z., Feng, Y., Weimar, W. R., Luchetta, G. R., Wu, Q., Ortiz-Ocasio, J., Vinson, J. R. T., Kramer, D. and Porter, C., J. Med. Chem, 1994, 3464-3476.
Christiansen, K., 1999, Int. J. Antimicrob. Agents, S73-S78.
Diassi, P. A., Bacso, I., Krakower, G. W., Ann Van Dine, H., 1966, Tetrahedron, 3459-3467.
Duvold, T., Sorensen, S. D., Björkling, F., Henriksen, A. S. and Rastrup-Andersen, N., 2001, J. Med. Chem., 44, 3125-3131.
Gaell, A. J., Blagbrough, I. S., 2000, Tetrahedron, 2449-2460.
Godtfredsen, W. O., Vangedal, S., 1962, Tetrahedron, 1029-1048.
Godtfredsen, W. O., Albrethsen, C., von Daehne, W., Tybring, L., Vangedal, S., 1965ₐ, Antimicrob. Agents Chemotherapy, 132-137.
Godtfredsen, W. O., von Daehne, W., Vangedal, S., Marquet, A., Arigoni, D., Melera, A., 1965_{b}, Tetrahedron, 3505-3530.
Godtfredsen, W. O., von Daehne, W., Tybring, L., Vangedal, S., 1966, J. Med. Chem., 15-22.
Goodnov, Jr., R., Konno, K., Niwa, M., Kallimopoulos, T., Bukownik, R., Lenares, D., Nakanishi, K., Tetrahedron, 1990, 3267-3286.
Hong-Seok Kim, H.-S., Bo-Seung Choi, B.-S., Kyung-Chan Kwon, K.-C., Sang-Ok Lee, S.-O, Hyun Jung Kwak, H. J., Cheol Hae Lee, C. H., 2000, Bioorg. Med. Chem., 2059-2065.
Karagiannis, G., Papaioannou, D., Eur. J. Org. Chem., 2000, 1841-1863.
Kikuchi, K., Bernard, E. M., Sadownik, A., Regen, S. L., Armstrong, D., 1997, Antimicrob. Agents Chemoterap., 1433-1438.
Kinney, W. A., Zhang, X., Williams, J. I., Johnston, S., Michalak, R. S., Deshpande, M., Dostal, L., Rosazza, J. P. N., 2000, Org. Lett., 2921-2922.
Kuchers A., Crove, S., Grayson, M. L., Hoy, J., in The Use of Antibiotics, 5.ed., Butterworth Heinemann, Oxford, 1997.
Kuksa, V., Buchan, R., Kong Thoo Lin, P., 2000, Synthesis, 1189-1207.
Moore, K. S., Wehrli, S., Roder, H., Rogers, M., Forrest, Jr., J. N., McCrimmon, D., Zasloff, M., 1993, Proc. Natl. Acad. Sci. USA, 1354-1358.
Pechulis, A. D., Bellevue III, F. H., Cioffi, C. L., Trapp, S. G., Fojtik, J. P., McKitty, A. A., Kinney, W. A., Frye, L. L., 1995, J. Org. Chem., 5121-5126.
Rao, M. N., Shinnar, A. E., Noecker, L. A., Chao, T. L., Feibush, B., Snyder, B., Sharkansky, I., Sarkahian, A, Zhang, X., Jones, S. R., Kinney, W. A., Zasloff, M., 2000, J. Nat. Prod., 631-635.
Sadownik, A., Deng, G., Janout, V., Regen, S. L., 1995, J. Am. Chem. Soc., 6138-6139.
Savage, P. B., Li, C., 2000, Exp. Opin. Invest. Drugs, 263-272.
Stromgaard, K., Brierley, M. J., Andersen, K., Slok, F. A., Mellor, I. R., Usherwood, P. N. R., Krogsgaard-Larsen, P., Jaroszewski, J. W., 1999, J. Med. Chem., 5224-5234.
von Daehne, W., Godtfredsen, W. O., Rasmussen, P., 1979, Adv. Appl. Microbiol., 95-145.
Weis, A. L., Bakos, T., Alferiev, I., Zhang, X., Shao, B., Kinney, W. A., 1999, Tetrahedron Lett., 4863-4864.

1. A compound according to formula I 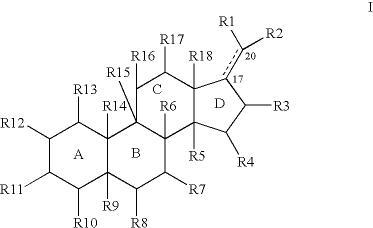
wherein the fused rings A, B, C and D are independently saturated or fully or partially unsaturated;
the bond between C-17 and C-20 is depicted with a full and a dotted line to indicate that said bond can be a single or a double bond;
wherein R1 is hydrogen, halogen, a lipophilic group, -(Z)ₙ-(NR-Z)ₚ-N(R)₂ or C(O)-(Z), —(NR-Z)ₚ-N(R)₂, wherein n is 0 or 1 and p is an integer from 1 and 5;
each Z independently represents straight or branched hydrocarbon diradical, optionally substituted with C₁₋₆ alkyl, C₁₋₆alkenyl, C₁₋₆alkynyl, hydroxy, alkoxy, amino, C₁₋₆aminoalkoxy, C₁₋₆aminoalkyl, C₁₋₆aminoalkylaminocarbonyl, C₁₋₆alkylC₃₋₈cycloalkyl or C₁₋₆alkylheteroaryl;
each R independently represents hydrogen or C₁₋₆alkyl, C I₆aminoalkyl,
C₁₋₆aminoalkoxy or C₁₋₆aminoalkylaminocarbonyl, all of which are optionally substituted with alkyl or C₁₋₆aminoalkyl;
provided that at least one Z is substituted with C₁₋₆ alkyl, C₁₋₆alkenyl, C₁₋₆alkynyl, hydroxy, alkoxy, C₁₋₆aminoalkoxy, C₁₋₆aminoalkyl, C₁₋₆aminoalkylaminocarbonyl, C₁₋₆alkylC₃₋₈cycloalkyl or C₁₋₆alkylheteroaryl, or at least one R is different from hydrogen;
R2 represents halogen, C₁₋₄alkyl, optionally substituted with COOH; C₁₋₄alkoxy, —COOH, -(Z)ₙ-(NR-Z)ₚ-N(R)₂ or C(O)-(Z)ₙ-(NR-Z)ₚ-N(R)₂;
R3 represents hydrogen halogen or O—R19, wherein R19 represents hydrogen, —SO₃, C₁₋₆alkyl, C₁₋₆acyl or -(Z)ₙ-(NR-Z)ₚ-N(R)₂;
each of R4, R7, R8, R11, R12, R13, R16 and R17 independently represent hydrogen, halogen, hydroxy, —OSO₃, —O-acyl, -(Z)ₙ-(NR-Z)ₚ-N(R)₂ or C(O)-(Z)ₙ-(NR-Z)ₚ-N(R)₂;
R10 represents hydrogen, methyl, halogen, hydroxy, —OSO₃, —O-acyl, -(Z)ₙ-(NR-Z)ₚ-N(R)₂ or C(O)-(Z)ₙ-(NR-Z)ₚ-N(R)₂;
each of R5, R6, R9, R14, R15 and R18 independently represent hydrogen or methyl or are each independently absent when one of the fused rings, A, B, C and D are unsaturated so as to complete the valency of the carbon atom at that site;
provided that at least one, and not more than three of R1, R2, R4, R7, R8, R10, R11, R12, R13, R16 and R17 is -(Z)ₙ-(NR-Z)ₚ-N(R)₂ or C(O)-(Z)ₙ-(NR-Z)ₚ-N(R)₂;
provided that the compound is not
3β-hydroxy-6β-(2-dimethylaminoethyl)amino-5α-stigmastane,
3β-hydroxy-6β-(2-diethylaminoethyl)amino-5α-stigmastane,
3β-hydroxy-6β-(3-dimethylaminopropyl)amino-5α-stigmastane,
3β-hydroxy-6α-(2-diethylaminoethyl)amino-5α-stigmastane,
3β-hydroxy-6β-(2-dimethylaminoethyl)amino-5α-cholestane,
3β-hydroxy-6β-(2-diethylaminoethyl)amino-5α-cholestane,
3β-hydroxy-6β-(3-dimethylaminopropyl)amino-5α-cholestane,
3β-hydroxy-6α-(2-diethylaminoethyl)amino-5α-cholestane,
20-(γ-diethylaminopropyl) amino-5α-pregnan-3β-ol,
20-(β-diethylaminoethyl)-amino-5α-pregnan-3β-ol,
20-(β-dimethyl aminoethyl)-amino-5α-pregnan-3β-ol,
20-(β-dimethylaminoethyl)-aminopregn-5-en-3β-ol,
20-(β-diethylaminoethyl)-aminopregn-5-en-3β-ol,
N(β-diethylaminoethyl)-3α,7α,12α-trihydroxy-5β-cholan-24-amide,
N(β-diethylaminoethyl)-3α,12α-dihydroxy-5β-cholan-24-amide,
N(β-diethylaminoethyl)-3α,7α,12α-trihydroxy-5β-cholan-24-amine, or
N(β-diethylaminoethyl)-3α,12α-dihydroxy-5β-cholan-24-amine, and
and pharmaceutically acceptable salts or esters thereof.

2. A compound according to claim 1, wherein R2 represents -(Z)ₙ-(NR-Z)ₚ-N(R)₂ or C(O)-(Z)ₙ-(NR-Z)ₚ-N(R)₂.

3. A compound according to claim 1, wherein R7, R11 and/or R16 represents -(Z)ₙ-(NR-Z)ₚ-N(R)₂ or C(O)-(Z), —(NR-Z)ₚ-N(R)₂.

4. A compound according to claim 1, wherein R1 represents a lipophilic group.

5. A compound according to claim 1, wherein R1 is selected from the group consisting of straight or branched, saturated or unsaturated C₁₋₁₀alkyl, aryl, C₃₋₈cycloalkyl, aralkyl with 1-10 carbon atoms in the alkyl moiety, C₁₋₁₀alkylaryl, C₁₋₁₀alkyl-C₃₋₈cycloalkyl, C₁₋₁₀alkoxy and heteroaryl.

6. A compound according to any of claims 1-5, wherein R19 represents C₁₋₆alkyl or C₁₋₆acyl.

7. A compound according to any of claims 16 claim 1, wherein R7, R11 and/or R16 represents OH

8. A compound according to any of claims 1-5, wherein R11 represents —OSO₃.

9. A compound according to any of claims 1-5, wherein R11 represents —O-acyl.

10. A compound according to claim 1 which has the general formula Ia

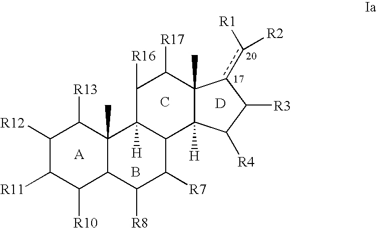

11. A compound according to claim 1 which has the general formula Ib

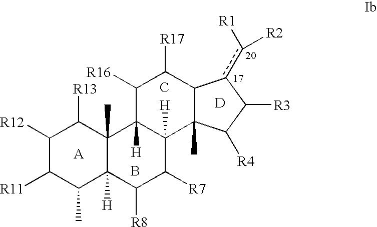

12. A compound according to claim 10 or 11, wherein R represents -(Z)ₙ-(NR-Z)ₚ-N(R)₂ or C(O)-(Z)ₙ-(NR-Z)ₚ-N(R)₂.

13. A compound according to claim 12, wherein R7 and R11 are both hydroxy.

14. A compound according to claim 12, wherein R11 and R16 are both hydroxy.

15. A compound according to claim 12, wherein R3 is —OR19, wherein R19 is C₁₋₆alkyl or C₁₋₆acyl.

16. A compound according to claim 12, wherein R1 is a lipophilic group.

17. A compound according to claim 12, wherein R1 is a straight or branched, saturated or unsaturated C₁₋₁₀hydrocarbon.

18. A compound according to claim 12, wherein R1 is a moiety of formula II 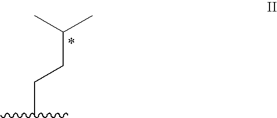
wherein the carbon-carbon bond denoted “*” is a single or double bond.

19. A compound according to claims 10 or 11, wherein R11 represents -(Z)ₙ-(NR-Z)ₚ-N(R)₂ or C(O)-(Z)ₙ-(NR-Z)ₚ-N(R)₂.

20. A compound according to claim 19, wherein R2 is C₁₋₄alkyl, optionally substituted with COOH, C₁₋₄alkoxy or COOH.

21. A compound according to claim 19, wherein R3 is O—R19, wherein R19 represents C₁₋₆alkyl or C₁₋₆acyl.

22. A compound according to claim 19, wherein R1 is a lipophilic group.

23. A compound according to claim 19, wherein R1 is a straight or branched, saturated or unsaturated C₁₋₁₀hydrocarbon.

24. A compound according to claim 19, wherein R1 is a moiety of formula II 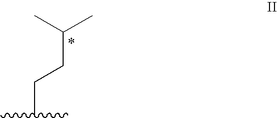
wherein the carbon-carbon bond denoted “*” is a single or double bond.

25. A compound according to any one of claims 1, 10 or 11, wherein R2 and/or R11 represents a moiety of the formula VIII, IX, X, XI, XII or XIII

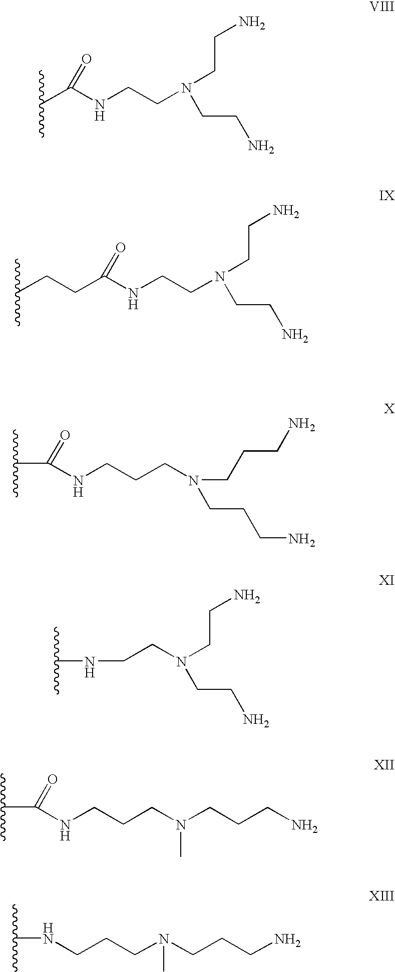

26. A compound according to claim 1 selected from the list consisting of
21-N-{2′-[bis(2′-aminoethyl)amino]ethyl}-17R,20S,24,25-tetrahydrofusid-21-amide,
21-N-{2′-[bis(2′-aminoethyl)amino]ethyl}-11-desoxy-17R,20S,24,25-tetrahydrofusid-21-amide,
21-N-{2′-[bis(2′-aminoethyl)amino]ethyl}-16-desacetoxy-17R,20S,24,25-tetrahydrofusid-21-amide,
21-N-{2′-[bis(2′-aminoethyl)amino]ethyl}-13(17)-en-17,20,24,25-tetrahydrofusidan-21-carboxamide,
21-N-{2′-[bis(2′-aminoethyl)amino]ethyl}-3β-desacetoxy-17R,20S,24,25-tetrahydrofusid-21-amide,
21-N-{2′-[bis(2′-aminoethyl)amino]ethyl}-9(11)-en-17R,20S,24,25-tetrahydrofusid-21-amide,
24-N-{2′-[bis(2′-aminoethyl)amino]ethyl}-3α-hydroxy-5β-cholan-24-amide,
22-N-{2′-[bis(2′-aminoethyl)amino]ethyl)-23,24-bisnor-5-cholenic-22-amide,
21-N-{2′-[bis(2′-aminoethyl)amino]ethyl)-fusid-21-amide,
21-N-{2′-[bis(3′-aminopropyl)amino]propyl}-fusid-21-amide, 21-N-{2′-[bis(2′-aminoethyl)amino]ethyl}-3-OSO₃-11-desoxy-17,20,24,25-tetrahydro-fusid-21-amide,
21-N-{2′-[bis(2′-aminoethyl)amino]ethyl}-11-desoxy-16-desacetoxy-17S,20,24,25-tetrahydrofusid-21-amide,
21-N-{3′-[bis(3′-aminopropyl)amino]propyl}-17R,20S,24,25-tetrahydrofusid-21-amide,
22-N-{3′-[bis(3′-aminopropyl)amino]propyl}-23,24-bisnor-5-cholenic-22-amide,
21-N-{3′-[bis(3′-aminopropyl)amino]propyl}-}-3-OAc-17R,20S,24,25-tetrahydrofusid-21-amide,
21-N-{3′-[bis(3′-aminopropyl)amino]propyl}-}-3-OSO₃-11-desoxy-17,20,24,25-tetrahydrofusid-21-amide,
21-N-{3′-[bis(3′-aminopropyl)amino]propyl}-}-11-desoxy-16-desacetoxy-17S,20,24,25-tetrahydrofuisid-21-amide,
3-N-{2′-[bis(2′-aminoethyl)amino]ethyl}-fusidic acid,
21-N-{3′-[(3′-aminopropyl)(methyl)amino]propyl}-17R,20S,24,25-tetrahydrofusid-21-amide,
21-N-{3′-[(3′-aminopropyl)(methyl)amino]propyl}-11-desoxy-17R,20S,24,25-tetrahydrofusid-21-amide,
21-N-{3′-[(3′-aminopropyl)(methyl)amino]propyl}-16-desacetoxy-17R,20S,24,25-tetrahydrofusid-21-amide,
24-N-{3′-[(3′-aminopropyl)(methyl)amino]propyl-3α-hydroxy-5β-cholan-24-amide,
21-N-{3′-[(3′-aminopropyl)(methyl)amino]propyl}-11 desoxy-16-desacetoxy-17R,20S,24,25-tetrahydrofusid-21-amide,
3-N-{3′-[bis(3′-aminopropyl)amino]propyl}-}-fusidic acid,
3-N-{3′-[(3′-aminopropyl)(methyl)amino]propyl}-fusidic acid.

27. A pharmaceutical composition comprising a compound according to claim 1, optionally together with a pharmaceutically acceptable excipient or vehicle, and optionally other therapeutically active agents.

28. A composition according to claim 27, wherein said other therapeutically active agent is selected from the group consisting of penicillins, cephalosporins, tetracyclines, rifamycins, erythromycins, lincomycin, clindamycin, flouroquinolones, corticosteroids, hydrocortosone and triamcinolone.

29. The use of a compound according to claim 1 for the manufacture of a medicament for the treatment of prevention of infections.

30. The use according to claim 29, wherein the infection is bacterial

31. The use according to claim 29, wherein said compound is combined with one or more other therapeutically active ingredients.

32. The use according to claim 29, wherein said compound is combined with one or more other compounds selected from the group consisting of penicillins, cephalosporins, tetracyclines, rifamycins, erythromycins, lincomycin, clindamycin, flouroquinolones, corticosteroids, hydrocortosone and triamcinolone.

33. A method of preventing or treating infection, the method comprising administering to a patient in need thereof an effective amount of a compound according to claim 1.

34. A method according to claim 33, wherein said infection is bacterial.

35. A method according to claim 33, wherein said compound is administered simultaneously or sequentially with one or more other therapeutically active agents.

36. A method according to claim 35, wherein said other terapeutically active agent is selected from the list consisting of penicillins, cephalosporins, tetracyclines, rifamycins, erythromycins, lincomycin, clindamycin, flouroquinolones, corticosteroids, hydrocortosone and triamcinolone.
